(19) United States
(12) Patent Application Publication
(10) Pub. No.: US 2017/0208817 A1
(43) Pub. Date: Jul. 27, 2017

(54) Isolated Bacterial Strain of the Genus Burkholderia and Pesticidal Metabolites Therefrom-Formulations and Uses

(71) Applicant: Marrone Bio Innovations, Inc., Davis, CA (US)

(72) Inventors: Ratnakar Asolkar, Davis, CA (US); Marja Koivunen, Davis, CA (US); Pamela Marrone, Davis, CA (US)

(21) Appl. No.: 15/481,511

(22) Filed: Apr. 7, 2017

Related U.S. Application Data
(63) Continuation of application No. 14/238,467, filed on Feb. 11, 2014.
(60) Provisional application No. 61/528,153, filed on Aug. 27, 2011.
(60) Provisional application No. 61/528,149, filed on Aug. 27, 2011.

(51) Int. Cl. A01N 63/02; A01N 31/02; A01N 43/16; C12P 7/62; A01N 43/90; A01N 43/76; A01N 63/00; A01N 37/40

(52) U.S. Cl. CPC A01N 63/02; A01N 63/00; A01N 31/02; A01N 37/40; C12P 7/62; A01N 43/90; A01N 43/76; A01N 43/16

(57) ABSTRACT

The present invention provides a method of the proliferation of pest infestation using a species of Burkholderia. Also provided are natural products derived from a culture of said species and methods of controlling algae and/or arachnids using said natural products.

### CROSS-REFERENCE TO RELATED APPLICATIONS

This patent application is a Continuation application of U.S. patent application Ser. No. 14/238,467, filed on Feb. 11, 2014 and entitled “Isolated Bacterial Strain of the Genus Burkholderia and Pesticidal Metabolites Therefrom—Formulations and Uses,” which claims priority to the International Application No. PCT/US2012/050807, filed on Aug. 14, 2012 and claims the priority of U.S. Provisional Patent Application Ser. No. 61/528,153, filed on Aug. 27, 2011 and U.S. Provisional Patent Application Ser. No. 61/528,149, filed on Aug. 27, 2011, the contents of which are incorporated by reference herein in their entirety.

### TECHNICAL FIELD OF THE INVENTION

Provided herein is a species of Burkholderia sp with no known pathogenicity to vertebrates, such as mammals, fish and birds but pesticidal activity against plants, algae, insects, fungi, arachnids, such as mites and nematodes and formulations and compositions comprising said species. Also provided are natural products, formulations and compositions derived from a culture of said species and methods of controlling algae and arachnids, such as mites, using said Burkholderia and/or said natural products.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Jan. 21, 2014, is named MBII1013CON_SeqList and is 14 kilobytes in size.

### BACKGROUND OF THE INVENTION

Natural products are substances produced by microbes, plants, and other organisms. Microbial natural products offer an abundant source of chemical diversity, and there is a long history of utilizing natural products for pharmaceutical purposes. One such compound is FR901228 isolated from Chromobacterium and has been found to be useful as an antibacterial agent and antitumor agent (see, for example, Ueda et al., U.S. Pat. No. 7,396,665).

However, secondary metabolites produced by microbes have also been successfully found to have uses for weed and pest control in agricultural applications (see, for example, Nakajima et al. 1991; Duke et al., 2000; Lydon & Duke, 1999; Gerwick et al., U.S. Pat. No. 7,393,812). Microbial natural products have been also successfully developed into agricultural insecticides (see, for example, Salama et al. 1981; Thompson et al., 2000; Krieg et al. 1983). Sometimes, such natural products have been combined with chemical pesticides (see, for example, Gottlieb, U.S. Pat. No. 4,808,207).

Acaricides are compounds that kill mites (miticides) and ticks (ixodicides). This class of pesticides is large and includes antibiotics, carbamates, formamidine acaricides, pyrethroids, mite growth regulators, and organophosphate acaricides. Besides chemical pesticides, diatomaceous earth and fatty acids can be used to control mites. They typically work through disruption of the cuticle, which dries out the mite. In addition, some essential oils such as peppermint oil, are used to control mites. In spite of the great variety of known acaricide compounds, mites remain a serious problem in agriculture because of the damage they cause to the crops. They can produce several generations during one season, which facilitates rapid development of resistance to the acaricide products used. Hence, new pesticide products with new target sites and novel modes of action are critically needed.

Algae come in many forms. These include: (1) microscopic, one-celled algae, filamentous algae that resemble hair, algae that grow in sheets and macroalgae that look like plants; (2) algae that live inside the outer integument (“skin”) or calcium shell of some corals, anemones, and other sessile invertebrates called zooxanthellae; (3) very hard-to-remove little dots of green that sometimes grow on aquarium panels which also are not algae, but diatom or radiolarian colonies (microscopic, one-celled, animals with hard shells) with algae incorporated in their matrix.

Growth of algae in a small amount of water retained in the container over a significant period of time can be considerable, which is highly undesirable. As a result, algae can cause clogging of filters in water filtration devices, undesirable smells and appearance in pools, exhaustion of dissolved oxygen, and suffocation of fishes and shellfishes to death. In addition to being present in water, algae may also be present in industrial materials which are exposed to the weather and light, such as coatings containing organic film formers on mineral substrates, textile finishes, wood paints and also materials made of plastics.

Algae control can be divided into four categories: biological, mechanical, physical and chemical controls. A few pertinent facts hold for all methods of algae control. For example, Turbo and Astrea snails, some blennies, some tangs, among others are good grazers. Snails are the most widely used scavengers, and generally the best choice. Some parts of the country seem to favor the use of sea urchins, dwarf angels. The former die too easily and move the decor about, and the latter can be problematical with eating expensive invertebrates. Other methods include functional protein skimmers, with or without ozone and ultraviolet sterilizers. These physical filters remove and destroy algae on exposure and help oxidize nutrients as the water is circulated. Antibiotics may also be used. However, they treat the symptoms only without dealing with the cause(s) of the algae problem. The factors can contribute to water system being out of balance. Copper, usually in the form of copper sulfate solution has been employed as an algicide, as well as a general epizootic parasite preventative. This metal is useful in treatment and quarantine tanks, dips and fish-only arrangements but it is persistent and toxic to all life, especially non-fish.

The Burkholderia genus, β-subdivision of the proteobacteria, comprises more than 40 species that inhabit diverse ecological niches (Compant et al., 2008). The bacterial species in the genus Burkholderia are ubiquitous organisms in soil and rhizosphere (Coenye and Vandamme, 2003; Parke and Gurian-Sherman, 2001). Traditionally, they have been known as plant pathogens, B. cepacia being the first one discovered and identified as the pathogen causing disease in onions (Burkholder, 1950). Several Burkholderia species have developed beneficial interactions with their plant hosts (see, for example, Cabballero-Mellado et al., 2004, Chen et al., 2007). Some Burkholderia species have also been found to be opportunistic human pathogens (see, for example, Cheng and Currie, 2005 and Nierman et al., 2004). Additionally, some Burkholderia species have been found to have potential as biocontrol products (see for example, Burkhead et al., 1994; Knudsen et al., 1987; Jansiewicz et al., 1988; Gouge et al., US Patent Application No. 2003/0082147; Parke et al., U.S. Pat. No. 6,077,505; Casida et al., U.S. Pat. No. 6,689,357; Jeddeloh et al., WO2001055398; Zhang et al., U.S. Pat. No. 7,141,407). Some species of in this genus have been effective in bioremediation to decontaminate polluted soil or groundwater (see, for example, Leahy et al. 1996). Further, some Burkholderia species have been found to secrete a variety of extracellular enzymes with proteolytic, lipolytic and hemolytic activities, as well as toxins, antibiotics, and siderophores (see, for example, Ludovic et al., 2007; Nagamatsu, 2001).

PCT/US2011/026016 discloses a Burkholderia species, particularly Burkholderia A396 and compounds derived from said species with no known pathogenicity to vertebrates with activity against plants, insects, fungi and nematodes.

Oxazoles, thiazoles and indoles are widely distributed in plants, algae, sponges, and microorganisms. A large number of natural products contain one or more of the five-membered oxazole, thiazole and indole nucleus/moieties. These natural products exhibit a broad spectrum of biological activity of demonstrable therapeutic value. For example, bleomycin A (Tomohisa et al.), a widely prescribed anticancer drug, effects the oxidative degradation of DNA and uses a bithiazole moiety to bind its target DNA sequences (Vanderwall et al., 1997). Bacitracin (Ming et al., 2002), a thiazoline-containing peptide antibiotic, interdicts bacterial cell wall new biosynthesis by complexation with C55-bactoprenolpyrophosphate. Thiangazole (Kunze et al., 1993) contains a tandem array of one oxazole and three thiazolines and exhibits antiviral activity (Jansen et al., 1992). Yet other oxazole/thiazole-containing natural products such as thiostrepton (Anderson et al., 1970) and GE2270A (Selva et al., 1997) inhibit translation steps in bacterial protein synthesis. More than 1000 alkaloids with the indole skeleton have been reported from microorganisms. One-third of these compounds are peptides with masses beyond 500 Da where the indole is tryptophan derived. The structural variety of the remaining two-thirds is higher, and their biological activity seems to cover a broader range, including antimicrobial, antiviral, cytotoxic, insecticidal, antithrombotic, or enzyme inhibitory activity.

### SUMMARY OF THE INVENTION

Provided herein is an isolated strain of a non-Burkholderia cepacia, non-Burkholderia plantari, non-Burkholderia gladioli, Burkholderia sp. In a particular embodiment, the strain has the identifying characteristics of a Burkholderia A396 strain (NRRL Accession No. B-50319).

The present invention provides a method of modulating the proliferation of pest infestation comprising the steps of: providing an insecticidal or herbicidal composition comprising: an isolated strain of Burkholderia sp. A 396 (NRRL Accession No. B-50319); n-hexanol; and hexyl paraben formed by incubating isolated strain of Burkholderia sp. A 396 (NRRL Accession No. B-50319) in n-hexanol; and applying the insecticidal or herbicidal composition to a location where modulation the proliferation of pests is desired an amount of affective to modulate the proliferation of the pest. The hexyl paraben may be present in the amount of about 0.01-5%, and the n-hexanol is present in the amount of about 0.001-10%. The pest may be a nematode, a monocot, a dicot, or sedge weeds. The isolated strain of Burkholderia sp. A 396 (NRRL Accession No. B-50319) may be lysed.

The present invention provides a method for obtaining a hexyl paraben composition comprising the steps of: providing a composition comprising an isolated strain of Burkholderia sp. A 396 (NRRL Accession No. B-50319); incubating the composition in n-hexanol for a time and at a temperature sufficient to produce said hexyl paraben composition. The hexyl paraben may be present in the amount of about 0.01-5%. The n-hexanol may be present in the amount of about 0.001-10%. The method to claim 13, wherein said hexyl paraben is present in the amount of about 0.01-5%. The composition may be may be a cell-free supernatant and/or the composition may include lysed Burkholderia sp. A 396 (NRRL Accession No. B-50319).

The hexyl paraben composition may be made in to a compostion for modulating the proliferation of a pest infestation by adding a carrier compatible with the purpose. a carrier to the hexyl paraben composition to form a

Also provided is a combination, particularly a composition or formulation comprising (a) a first substance selected from the group consisting of a pure culture, cell fraction or supernatant derived from the Burkholderia strain set forth above or extract thereof for use optionally as a pesticide; and (b) optionally at least one of a carrier, diluent, surfactant, adjuvant, or chemical or biological pesticide (e.g., algicide, acaricide, herbicide, fungicide, insecticide, nematocide and particularly, algicide or acaricide (e.g., miticide)). In a related aspect, provided herein is a seed coated with said combination or composition.

In a related aspect, disclosed is a method for modulating proliferation and/or growth of a pest including but not limited to insect, fungi, weeds, nematode, arachnid, algae and particularly, algae, arachnid (e.g., mites. ticks) comprising applying to a location where modulation of proliferation and/or growth of a pest is desired an amount of: (I)(a) at least one or more substances selected from the group consisting of a substantially pure cell culture, cell fraction, supernatant derived from the Burkholderia strain set forth above or extract thereof and (b) optionally another substance, wherein said substance is a pesticide, or (II) the combination, composition or formulation or pesticidal substances derived from said formulation set forth above, effective to modulate proliferation and/or growth of a pest at said location.

Disclosed herein are isolated compounds which are optionally obtainable or derived from Burkholderia species, or alternatively, organisms capable of producing these compounds that can be used to control various pests, particularly plant phytopathogenic pests, examples of which include but are not limited to insects, nematodes, bacteria, fungi. These compounds may also be used as herbicides, acaricides or algicides.

### BRIEF DESCRIPTION OF THE DRAWINGS

For a more complete understanding of the features and advantages of the present invention, reference is now made to the detailed description of the invention along with the accompanying figures and in which:

FIG. 1 shows the comparison of the growth rate of Burkholderia A396 to Burkholderia multivorans ATCC 17616.

FIG. 2 shows the general scheme used to obtain fractions from formulated MBI-206.

FIG. 3 shows the general scheme used to obtain fractions and compounds from an MBI-206 culture.

FIG. 4 shows insecticidal (sucking) activities of tested compounds against milkweed bugs (Oncopeltus fasciatus).

FIG. 5 shows insecticidal (feeding) activities of pure compounds against Lygus Hesperus.

### DETAILED DESCRIPTION OF THE INVENTION

While the compositions and methods heretofore are susceptible to various modifications and alternative forms, exemplary embodiments will herein be described in detail. It should be understood, however, that there is no intent to limit the invention to the particular forms disclosed, but on the contrary, the intention is to cover all modifications, equivalents, and alternatives falling within the spirit and scope of the invention as defined by the appended claims.

Where a range of values is provided, it is understood that each intervening value, to the tenth of the unit of the lower limit unless the context clearly dictates otherwise, between the upper and lower limit of that range and any other stated or intervening value in that stated range, is included therein. Smaller ranges are also included. The upper and lower limits of these smaller ranges are also included therein, subject to any specifically excluded limit in the stated range.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

As defined herein, “derived from” means directly isolated or obtained from a particular source or alternatively having identifying characteristics of a substance or organism isolated or obtained from a particular source.

As defined herein, an “isolated compound” is essentially free of other compounds or substances, e.g., at least about 20% pure, preferably at least about 40% pure, more preferably about 60% pure, even more preferably about 80% pure, most preferably about 90% pure, and even most preferably about 95% pure, as determined by analytical methods, including but not limited to chromatographic methods, electrophoretic methods.

As used herein, the term “alkyl” refers to a monovalent straight or branched chain hydrocarbon group having from one to about 12 carbon atoms, including methyl, ethyl, n-propyl, isopropyl, n-butyl, isobutyl, tert-butyl, n-hexyl, and the like.

As used herein, “substituted alkyl” refers to alkyl groups further bearing one or more substituents selected from hydroxy, alkoxy, mercapto, cycloalkyl, substituted cycloalkyl, heterocyclic, substituted heterocyclic, aryl, substituted aryl, heteroaryl, substituted heteroaryl, aryloxy, substituted aryloxy, halogen, cyano, nitro, amino, amido, —C(O)H, acyl, oxyacyl, carboxyl, sulfonyl, sulfonamide, sulfuryl, and the like.

As used herein, “alkenyl” refers to straight or branched chain hydrocarbyl groups having one or more carbon-carbon double bonds, and having in the range of about 2 up to 12 carbon atoms, and “substituted alkenyl” refers to alkenyl groups further bearing one or more substituents as set forth above.

As used herein, “alkynyl” refers to straight or branched chain hydrocarbyl groups having at least one carbon-carbon triple bond, and having in the range of about 2 up to 12 carbon atoms, and “substituted alkynyl” refers to alkynyl groups further bearing one or more substituents as set forth above.

As used herein, “aryl” refers to aromatic groups having in the range of 6 up to 14 carbon atoms and “substituted aryl” refers to aryl groups further bearing one or more substituents as set forth above.

As used herein, “heteroaryl” refers to aromatic rings containing one or more heteroatoms (e.g., N, O, S, or the like) as part of the ring structure, and having in the range of 3 up to 14 carbon atoms and “substituted heteroaryl” refers toheteroaryl groups further bearing one or more substituents as set forth above.

As used herein, “alkoxy” refers to the moiety —O-alkyl-, wherein alkyl is as defined above, and “substituted alkoxy” refers to alkoxyl groups further bearing one or more substituents as set forth above.

As used herein, “thioalkyl” refers to the moiety —S-alkyl-, wherein alkyl is as defined above, and “substituted thioalkyl” refers to thioalkyl groups further bearing one or more substituents as set forth above.

As used herein, “cycloalkyl” refers to ring-containing alkyl groups containing in the range of about 3 up to 8 carbon atoms, and “substituted cycloalkyl” refers to cycloalkyl groups further bearing one or more substituents as set forth above.

As used herein, “heterocyclic”, refers to cyclic (i.e., ring-containing) groups containing one or more heteroatoms (e.g., N, O, S, or the like) as part of the ring structure, and having in the range of 3 up to 14 carbon atoms and “substituted heterocyclic” refers to heterocyclic groups further bearing one or more substituent's as set forth above.

As used herein “algae” refers to any of various chiefly aquatic, eukaryotic, photosynthetic organisms, ranging in size from single-celled forms to the giant kelp. The term may further refer to photosynthetic protists responsible for much of the photosynthesis on Earth. As a group, the algae are polyphyletic. Accordingly, the term may refer to any protists considered to be algae from the following groups, alveolates, chloraraachniophytes, cryptomonads, euglenids, glaucophytes, haptophytes, red algae such as Rhodophyta, stramenopiles, and viridaeplantae. The term refers to the green, yellow-green, brown, and red algae in the eukaryotes. The term may also refer to the cyanobacteria in the prokaryotes. The term also refers to green algae, blue algae, and red algae.

As used herein “algicide” refers to one or more agents, compounds and/or compositions having algaestatic and/or algaecidal activity.

As used herein “algicidal” as used herein means the killing of algae.

As used herein “algistatic” as used herein means inhibiting the growth of algae, which can be reversible under certain conditions.

The Burkholderia strain set forth herein is a non-Burkholderia cepacia complex, non-Burkholderia plantari, non-Burkholderia gladioli, Burkholderia sp and non-pathogenic to vertebrates, such as birds, mammals and fish. This strain may be isolated from a soil sample using procedures known in the art and described by Lorch et al., 1995. The Burkholderia strain may be isolated from many different types of soil or growth medium. The sample is then plated on potato dextrose agar (PDA). The bacteria are gram negative, and it forms round, opaque cream-colored colonies that change to pink and pinkish-brown in color and mucoid or slimy over time.

Colonies are isolated from the potato dextrose agar plates and screened for those that have biological, genetic, biochemical and/or enzymatic characteristics of the Burkholderia strain of the present invention set forth in the Examples below. In particular, the Burkholderia strain has a 16S rRNA gene comprising a forward sequence that is at least about 99.5%, more preferably about 99.9% and most preferably about 100% identical to the sequence set forth in SEQ ID NO: 8, 11 and 12 and a forward sequence that is at least about 99.5%, more preferably about 99.9% and most preferably about 100% identical to the sequence set forth in SEQ ID NO: 9, 10, 13, 14 and 15 as determined by clustal analysis. Furthermore, as set forth below, this Burkholderia strain may, as set forth below, have pesticidal activity, particularly, virucidal, herbicidal, germicidal, fungicidal, nematicidal, bactericidal and insecticidal and more particularly, herbicidal, algicidal, acaricidal, insecticidal, fungicidal and nematicidal activity. It is not pathogenic to vertebrate animals, such as mammals, birds, and fish.

Additionally, the Burkholderia strain produces at least the pesticidal compounds set forth in the instant disclosure.

The Burkholderia strain is susceptible to kanamycin, chloramphenicol, ciprofloxacin, piperacillin, imipenem, and a combination of sulphamethoxazole and trimethoprim and contains the fatty acids 16:0, cyclo 17:0, 16:0 3-OH, 14:0, cyclo 19:0, 18:0.

This Burkholderia strain may be obtained by culturing a microorganism having the identifying characteristics of Burkholderia A396 (NRRL Accession No. B-50319) on Potato Dextrose Agar (PDA) or in a fermentation medium containing defined carbon sources such as glucose, maltose, fructose, galactose, and undefined nitrogen sources such as peptone, tryptone, soytone, and NZ amine.

The algicidal and acaricidal compounds disclosed herein may have the following properties: (a) is obtainable from a novel Burkholderia species, e.g., A396; (b) is, in particular, toxic to most common agricultural insect pests; (c) has a molecular weight of about 525-555 and more particularly, 540 as determined by Liquid Chromatography/Mass Spectroscopy (LC/MS); (d) has 1H NMR values of 6.22, 5.81, 5.69, 5.66, 5.65, 4.64, 4.31, 3.93, 3.22, 3.21, 3.15, 3.10, 2.69, 2.62, 2.26, 2.23. 1.74, 1.15, 1.12, 1.05, 1.02; (d) has 13C NMR values of 172.99, 172.93, 169.57, 169.23, 167.59, 130.74, 130.12, 129.93, 128.32, 73.49, 62.95, 59.42, 57.73, 38.39, 38.00, 35.49, 30.90, 30.36, 29.26, 18.59, 18.38, 18.09, 17.93, 12.51 (e) has an High Pressure Liquid Chromatography (HPLC) retention time of about 10-15 minutes, more specifically about 12 minutes and even more specifically about 12.14 min on a reversed phase C-18 HPLC (Phenomenex, Luna 5μ C18 (2) 100 A, 100×4.60 mm) column using a water:acetonitrile (CH3CN) with a gradient solvent system (0-20 min 90-0% aqueous CH3CN, 20-24 min 100% CH3CN, 24-27 min, 0-90% aqueous CH3CN, 27-30 min 90% aqueous CH3CN) at 0.5 mL/min flow rate and UV detection of 210 nm (f) has a molecular formula, C24H36N4O6S2, which is determined by interpretation of 1H, 13C NMR and LC/MS data (g) a 13C NMR spectrum with signals for all 24 carbons, including 5 methyl, 4 methylene, 9 methine, and 6 quaternary carbons and (g) 1H NMR spectrum displaying characteristics of a typical depsipeptide, illustrating three -amino protons [4.63, 4.31, 3.93], and one ester carbinol proton [5.69]. In a particular embodiment, the compound has the structure ##STR001##:

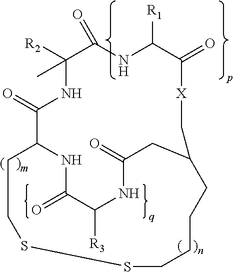

Or a pesticidally acceptable salt or stereoisomers thereof, wherein M is 1, 2, 3 or 4; n is 0, 1, 2, or 3; p and q are independently 1 or 2; X is O, NH or NR; R1, R2 and R3 are the same or different and independently an amino acid side-chain moiety or an amino acid side-chain derivative and R is a lower chain alkyl, aryl or arylalkyl moiety.

In an even more particular embodiment, the compound has the structure of FR901228:

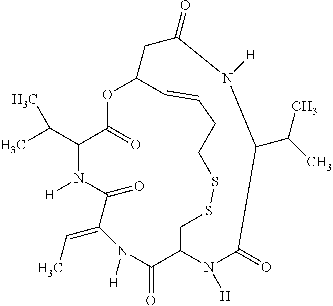 [SMILES: C/C=C1\NC(=O)C2CSSCC/C=C/C(CC(=O)NC(C(C)C)C(=O)N2)OC(=O)C(C(C)C)NC1=O]

Provided herewith are compounds set forth in ##STR002##:

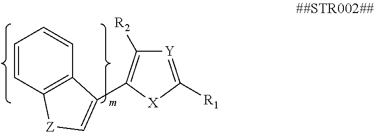

wherein: X, Y and Z are each independently —O, —NR₁, or —S, wherein R₁ is —H or C₁-C₁₀ alkyl; R_{I}, R₂ and m are each independently —H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl.

In an even another particular embodiment, Family ##STR002## compounds may be the compounds set forth in (vi)-(xix).

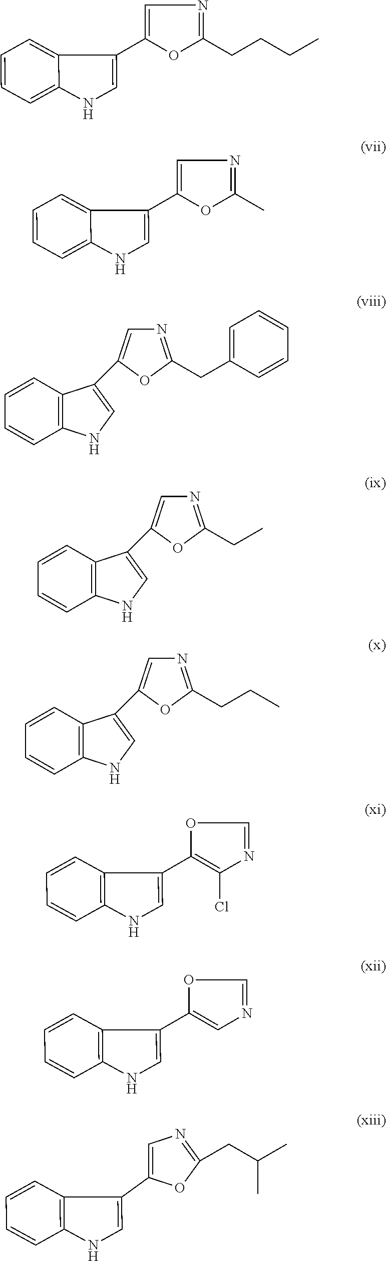

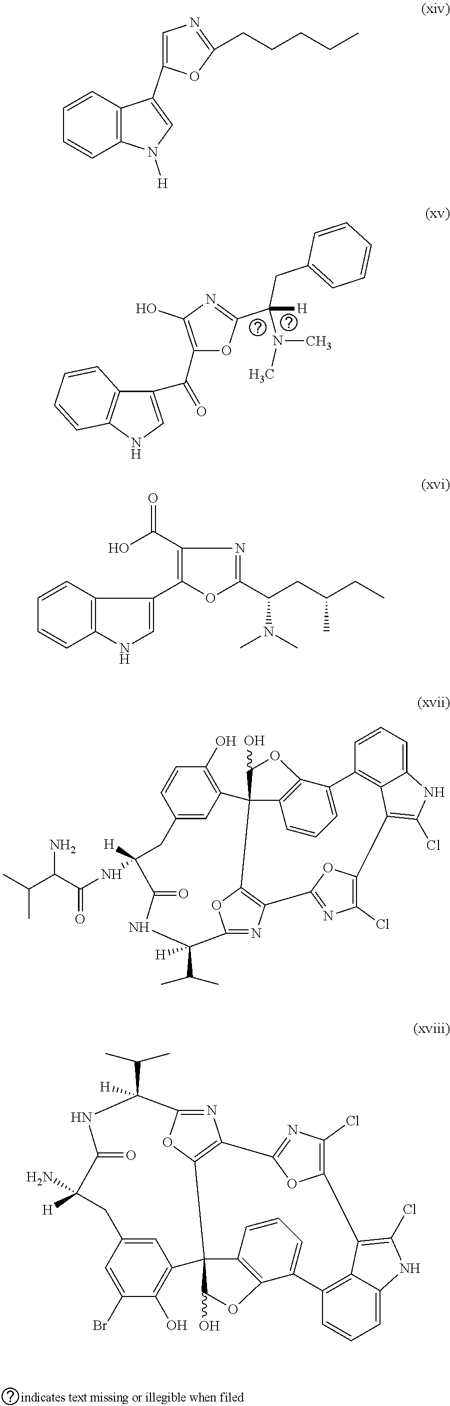

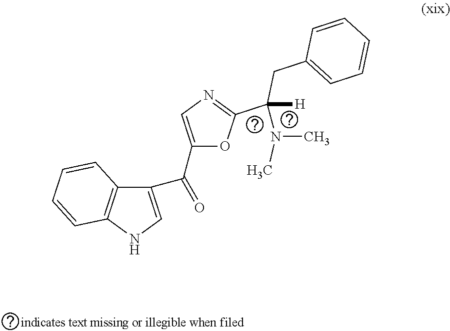 [SMILES: CN(C)[C@@H](Cc1ccccc1)c1ncc(C(=O)c2c[nH]c3ccccc23)o1]

These are from either natural materials or compounds obtained from commercial sources or by chemical synthesis. Natural sources of Family ##STR002## compounds include, but are not limited to, microorganisms, alga, and sponges. In a more particular embodiment, microorganisms which include the Family ##STR002## compounds include but are not limited to, or alternatively, Family ##STR002## compounds may be derived from species such as Streptoverticillium waksmanii (compound vi) (Umehara, et al., 1984), Streptomyces pimprina (compound vii) (Naiket al., 2001), Streptoverticillium olivoreticuli (compounds viii, ix, x) (Koyama Y., et al., 1981), Streptomyces sp (compounds xi, xii) (Watabe et al., 1988), Pseudomonas syringae (compounds xiii, xiv) (Pettit et al., 2002). Family ##STR002## compounds may also be derived from algae including but not limited to red alga (compound xv) (N'Diaye, et al., 1996), red alga Martensia fragilis (compound xvi) (Takahashi S. et al., 1998), Diazona chinensis (compounds xvii & xviii) (Lindquist N. et al., 1991), Rhodophycota haraldiophyllum sp (compound xix) (Guella et al., 1994).

Also provided is ##STR003##:

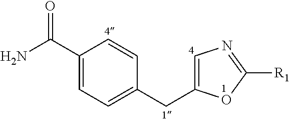

wherein: X and Y are each independently —OH, —NR₁, or —S, wherein R₁ is —H or C₁-C₁₀ alkyl; R_{I}, R₂ and m, a substituent on the oxazole ring, are each independently —H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl.

Further provided is ##STR005##:

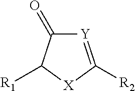

wherein X and Y are each independently —OH, —NR₁, or —S, wherein R₁, R₂ are each independently —H, alkyl (e.g., C₁-C₁₀ alkyl), substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl.

In a particular embodiment, Family ##STR005## compounds such as compounds from xx-xxiii set forth below may be derived from natural or commercial sources or by chemical synthesis.

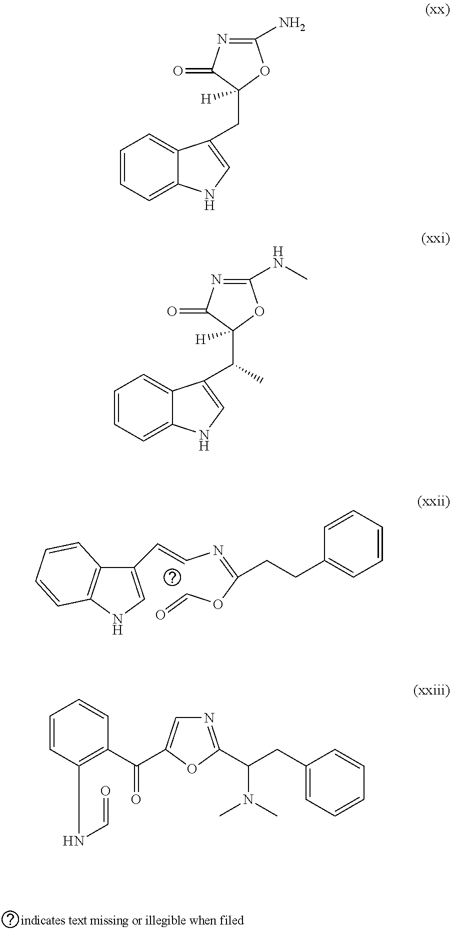 [SMILES: NC1=NC(=O)[C@H](Cc2c[nH]c3ccccc23)O1 ; CNC1=NC(=O)[C@H]([C@H](C)c2c[nH]c3ccccc23)O1 ; O=CO/C(CCc1ccccc1)=N\C=C\c1c[nH]c2ccccc12 ; CN(C)C(Cc1ccccc1)c1ncc(C(=O)c2ccccc2NC=O)o1]

Natural sources of Family ##STR005## compounds include, but are not limited to plants, corals, microorganisms, and sponges. The microorganisms include, but are not limited to Streptomyces griseus (compound xx) (Hirota et al., 1978), Streptomyces albus (compound xxi) (Werner et al., 1980). Family STR004 compounds may also be derived from algae including but not limited to Haraldiophyllum sp (compound xxii (Guella et al., 2006), and red algae (compound xxiii) (N'Diaye et al., 1994).

In one embodiment, the compound may be derived from or is obtainable from a microorganism, and in particular from Burkholderia species and characterized as having a structure comprising at least one ester, at least one amide, at least three methylene groups, at least one tetrahydropyranose moiety and at least three olefinic double bonds, at least six methyl groups, at least three hydroxyl groups, at least twenty five carbons and at least eight oxygen and one nitrogen. The compound further comprises at least one of the following characteristics: (a) pesticidal properties and in particular, nematicidal, fungicidal, insecticidal, acaricidal, algicidal and herbicidal properties; (b) a molecular weight of about 530-580 and more particularly, 555 as determined by Liquid Chromatography/Mass Spectroscopy (LC/MS); (c) 1H NMR values of δ 6.40, 6.39, 6.00, 5.97, 5.67, 5.54, 4.33, 3.77, 3.73, 3.70, 3.59, 3.47, 3.41, 2.44, 2.35, 2.26, 1.97, 1.81, 1.76, 1.42, 1.37, 1.16, 1.12, 1.04; (d) 13C NMR values of δ 173.92, 166.06, 145.06, 138.76, 135.71, 129.99, 126.20, 123.35, 99.75, 82.20, 78.22, 76.69, 71.23, 70.79, 70.48, 69.84, 60.98, 48.84, 36.89, 33.09, 30.63, 28.55, 25.88, 20.37, 18.11, 14.90, 12.81, 9.41; (e) an High Pressure Liquid Chromatography (HPLC) retention time of about 7-12 minutes, more specifically about 10 minutes and even more specifically about 10.98 min on a reversed phase C-18 HPLC (Phenomenex, Luna 5μ C18(2) 100 A, 100×4.60 mm) column using a water:acetonitrile (CH3CN) with a gradient solvent system (0-20 min; 90-0% aqueous CH3CN, 20-24 min; 100% CH3CN, 24-27 min; 0-90% aqueous CH3CN, 27-30 min; 90% aqueous CH3CN) at 0.5 mL/min flow rate and UV detection of 210 nm; (f) 13C NMR spectrum which exhibits 28 discrete carbon signals which may be attributed to six methyls, four methylene carbons, and thirteen methines including five sp2, four quaternary carbons; (g) a molecular formula of C28H45NO10 which was determined by interpretation of the ESIMS and NMR data analysis; (h) UV absorption bands between about 210-450 nm and most particularly at about 234 nm.

Also provided are compounds having the structure ##STR004a##:

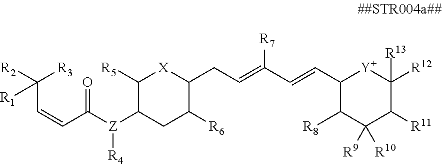

wherein X, Y and Z are each independently —O, —NR, or —S, wherein R is H or C₁-C₁₀alkyl; R₁, R₂, R₃, R₄, R₅, R₆, R₇, R₈, R₉, R₁₀, R₁₁, R₁₂, and R₁₃ are each independently H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl.

In a particular embodiment, the compound has the structure set forth in ##STR004b##:

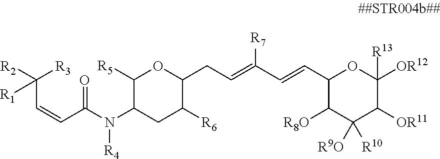

wherein R₁, R₂, R₃, R₄, R₅, R₆, R₇, R₈, R₉, R₁₀, R₁₁, R₁₂, and R₁₃ are as previously defined for ##STR004a##.

In a more particular embodiment, the compound is Templamide A with the following structure:

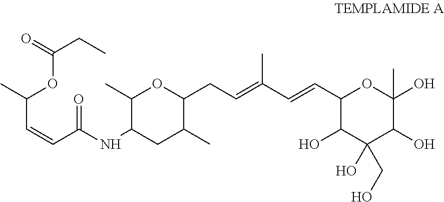 [SMILES: CCC(=O)OC(C)/C=C\C(=O)NC1CC(C)C(C/C=C(C)/C=C/C2OC(C)(O)C(O)C(O)(CO)C2O)OC1C]

In another embodiment, provided is a compound having formula ##STR004c##:

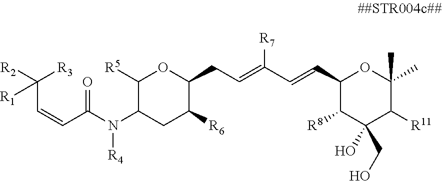

wherein R₁, R₂, R₃, R₄, R₅, R₆, R₇, R₈, and R₁₁ are as previously defined for ##STR004a##.

In another embodiment, provided is a compound which may be derived from Burkholderia species and characterized as having a structure comprising at least one ester, at least one amide, an epoxide methylene group, at least one tetrahydropyranose moiety and at least three olefinic double bonds, at least six methyl groups, at least three hydroxyl groups, at least 25 carbons and at least 8 oxygen and 1 nitrogen, and pesticide activity. The compound further comprises at least one of the following characteristics: (a) pesticidal properties and in particular, insecticidal, fungicidal, nematocidal, acaricidal, algicidal and herbicidal properties; (b) a molecular weight of about 520-560 and particularly 537 as determined by Liquid Chromatography/Mass Spectroscopy (LC/MS); (c) 1H NMR δ values at about 6.41, 6.40, 6.01, 5.97, 5.67, 5.55, 4.33, 3.77, 3.75, 3.72, 3.64, 3.59, 3.54, 3.52, 2.44, 2.34, 2.25, 1.96, 1.81, 1.76, 1.42, 1.38, 1.17, 1.12, 1.04; (d) 13C NMR values of δ 174.03, 166.12, 143.63, 137.50, 134.39, 128.70, 126.68, 124.41, 98.09, 80.75, 76.84, 75.23, 69.87, 69.08, 68.69, 68.60, 48.83, 41.07, 35.45, 31.67, 29.19, 27.12, 24.55, 19.20, 18.95, 13.48, 11.39, 8.04; (e) High Pressure Liquid Chromatography (HPLC) retention time of about 6-15 minutes, more specifically about 8 minutes on a reversed phase C-18 HPLC column using a water:acetonitrile (CH₃CN) gradient, particularly, an High Pressure Liquid Chromatography (HPLC) retention time of about 8-15 minutes, more specifically about 11 minutes and even more specifically about 11.73 min on a reversed phase C-18 HPLC (Phenomenex, Luna 5μ C18(2) 100 A, 100×4.60 mm) column using a water:acetonitrile (CH3CN) with a gradient solvent system (0-20 min; 90-0% aqueous CH3CN, 20-24 min; 100% CH3CN, 24-27 min; 0-90% aqueous CH3CN, 27-30 min; 90% aqueous CH3CN) at 0.5 mL/min flow rate and UV detection of 210 nm; (f) a molecular formula of C28H43NO9 which was determined by interpretation of the ESIMS and NMR data analysis;

(g) UV absorption bands at about 210-450 nm and most particularly at about 234 nm.

In a particular embodiment, the compound has the structure ##STR006a##:

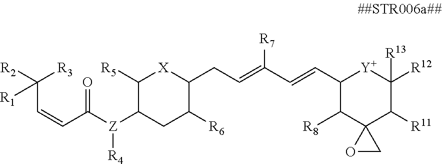

wherein X, Y and Z are each independently —O, —NR, or —S, wherein R is H or C₁-C₁₀ alkyl; R₁, R₂, R₃, R₄, R₅, R₆, R₇, R₈, R₁₁, R₁₂, and R₁₃ are each independently H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl.

In a particular embodiment, the compound has the structure:

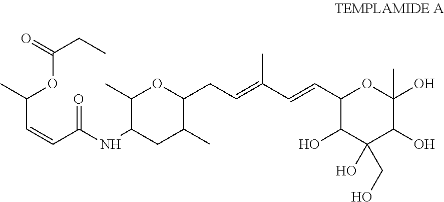 [SMILES: CCC(=O)OC(C)/C=C\C(=O)NC1CC(C)C(C/C=C(C)/C=C/C2OC(C)(O)C(O)C(O)(CO)C2O)OC1C]

In another embodiment, provided is a compound having formula ##STR006b##:

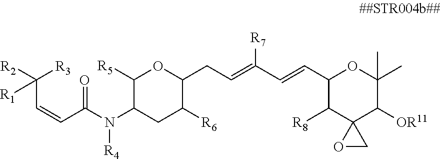

wherein R₁, R₂, R₃, R₄, R₅, R₆, R₇, R₈, and R₁₁ are as previously defined for ##STR006a##.

In a more particular embodiment, the compound is Templamide B with the following structure:

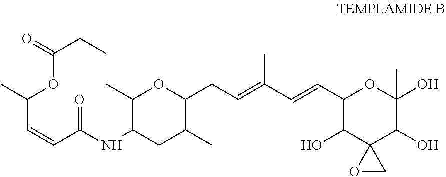 [SMILES: CCC(=O)OC(C)/C=C\C(=O)NC1CC(C)C(C/C=C(C)/C=C/C2OC(C)(O)C(O)C3(CO3)C2O)OC1C]

In yet another particular embodiment, the compound may be derived from Burkholderia species and characterized as having a structure comprising at least one ester, at least one amide, an epoxide methylene group, at least one tetrahydropyranose moiety and at least three olefinic double bonds, at least six methyl groups, at least three hydroxyl groups, at least 25 carbons and at least 8 oxygen and at least 1 nitrogen. The compound further comprises at least one of the following characteristics: (a) pesticidal properties and in particular, insecticidal, fungicidal, acaricidal, nematicidal, algicidal and herbicidal properties; (b) a molecular weight of about 510-550 and particularly about 523 as determined by Liquid Chromatography/Mass Spectroscopy (LC/MS); (c) 1H NMR δ values at about 6.41, 6.40, 6.01, 5.98, 5.68, 5.56, 4.33, 3.77, 3.75, 3.72, 3.65, 3.59, 3.55, 3.50, 2.44, 2.26, 2.04, 1.96, 1.81, 1.75, 1.37, 1.17, 1.04; (d) 13C NMR values of δ 172.22, 167.55, 144.98, 138.94, 135.84, 130.14, 125.85, 123.37, 99.54, 82.19, 78.28, 76.69, 71.31, 70.13, 69.68, 48.83, 42.52, 36.89, 33.11, 30.63, 25.99, 21.20, 20.38, 18.14, 14.93, 12.84; (e) an High Pressure Liquid Chromatography (HPLC) retention time of about 6-15 minutes, more specifically about 8 minutes on a reversed phase C-18 HPLC column using a water:acetonitrile (CH3CN) gradient, particularly, an High Pressure Liquid Chromatography (HPLC) retention time of about 8-15 minutes, more specifically about 10 minutes and even more specifically about 10.98 min on a reversed phase C-18 HPLC (Phenomenex, Luna 5μ C18(2) 100 A, 100×4.60 mm) column using a water:acetonitrile (CH3CN) with a gradient solvent system (0-20 min; 90-0% aqueous CH3CN, 20-24 min; 100% CH3CN, 24-27 min; 0-90% aqueous CH3CN, 27-30 min; 90% aqueous CH3CN) at 0.5 mL/min flow rate and UV detection of 210 nm; (f) a molecular formula of C27H41NO9 which was determined by interpretation of the ESIMS and NMR data analysis; (g) UV absorption bands at about 210-450 nm and most particularly at about 234 nm.

In a more particular embodiment, the compound is a known compound FR901465 which was isolated earlier from culture broth of a bacterium of Pseudomonas sp. No. 2663 (Nakajima et al. 1996) and had been reported to have anticancer activity with the following structure:

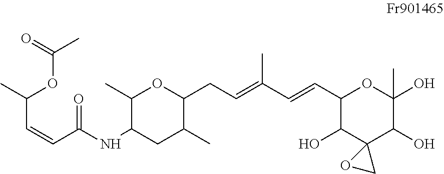 [SMILES: CC(=O)OC(C)/C=C\C(=O)NC1CC(C)C(C/C=C(C)/C=C/C2OC(C)(O)C(O)C3(CO3)C2O)OC1C]

In an even another particular embodiment, Family ##STR006a## compounds may be the compounds set forth in xxiv to xxxix. These are from either natural materials or compounds obtained from commercial sources or by chemical synthesis. Natural sources of Family ##STR006a## compounds include, but are not limited to, microorganisms, alga, and sponges. In a more particular embodiment, microorganisms which include the Family ##STR006a## compounds which may be derived from species such as Pseudomonas sp. No. 2663 (compounds xxiv-xxvi) (Nakajima et al., 1996), The synthetic analogues of the FR901464 (xxvii-xxxix) which have been synthesized and patented as anticancer compounds (see Koide et al., US Patent Application No. 2008/0096879 A1).

Also provided are the pesticidal compounds produced by the formulation set forth above which comprises at least one of the following characteristics: (a) has pesticidal properties and in particular, herbicidal, insecticidal, nematicidal, and fungicidal properties; (b) has a molecular weight of about 210-240 and more particularly, 222 as determined by Liquid Chromatography/Mass Spectroscopy (LC/MS); (e) has 1H NMR values of δ 7.90, 6.85, 4.28, 1.76, 1.46, 1.38, 1.37, 0.94; (d) has 13C NMR values of δ 166.84, 162.12, 131.34 (2C), 121.04, 114.83 (2C), 64.32, 31.25, 28.43, 25.45, 22.18. 12.93; (e) has an High Pressure Liquid Chromatography (HPLC) retention time of about 15-20 minutes, more specifically about 17 minutes and even more specifically about 17.45 min on a reversed phase C-18 HPLC (Phenomenex, Luna 5μ C18(2) 100 A, 100×4.60 mm) column using a water:acetonitrile (CH3CN) with a gradient solvent system (0-20 min; 90-0% aqueous CH3CN, 20-24 min; 100% CH3CN, 24-27 min; 0-90% aqueous CH3CN, 27-30 min; 90% aqueous CH3CN) at 0.5 mL/min flow rate and UV detection of 210 nm; (f) The 13C NMR spectrum exhibited 13 discrete carbon signals which were attributed to one methyl, five methylene carbons, four methines, and three quaternary carbons; (g) has a molecular formula of C13H18O3 which was determined by interpretation of the ESIMS and NMR data analysis; (h) has UV absorption bands between about 210-450 nm and most particularly at about 248 nm.

Also provided are compounds having the structure shown below

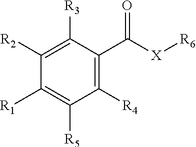

wherein X, is independently —O, —NR, or —S, wherein R is H or C₁-C₁₀ alkyl; R₁, R₂, R₃, R₄, R₅, and R₆ are each independently H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl.

In a more particular embodiment, the compound is butyl parben with the following structure:

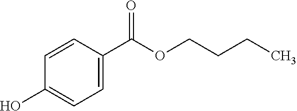

In a more particular embodiment, the compound is hexyl parben with the following structure:

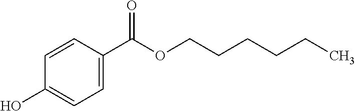 [SMILES: CCCCCCOC(=O)c1ccc(O)cc1]

In a more particular embodiment, the compound is octyl parben with the following structure:

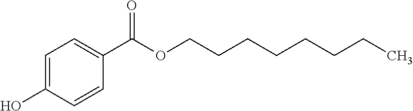 [SMILES: CCCCCCCCOC(=O)c1ccc(O)cc1]

In yet another embodiment, the compound is F7H18, which has a molecular weight of about 1080.

### Compositions

A substantially pure culture, cell fraction or supernatant and compounds produced by the Burkholderia strain disclosed herein, all of which are alternatively referred to as “active ingredient(s)”, may be formulated into pesticidal compositions. In a particular embodiment, the supernatant may be a cell-free supernatant.

The active ingredient(s) set forth above can be formulated in any manner. Non-limiting formulation examples include but are not limited to emulsifiable concentrates (EC), wettable powders (WP), soluble liquids (SL), aerosols, ultra-low volume concentrate solutions (ULV), soluble powders (SP), microencapsulation, water dispersed granules, flowables (FL), microemulsions (ME), nano-emulsions (NE), dusts, emulsions, liquids, flakes etc. In any formulation described herein, percent of the active ingredient is within a range of 0.01% to 99.99%.

A solid composition can be prepared by suspending a solid carrier in a solution of pesticidal compounds and drying the suspension under mild conditions, such as evaporation at room temperature or vacuum evaporation at 65° C. or lower. Alternatively, a solid composition may be derived via spray-drying or freeze-drying.

When referring to solid compositions, it should be understood by the artisan of ordinary skill that physical forms such as dusts, beads, powders, particulates, pellets, tablets, agglomerates, granules, floating solids and other known solid formulations are included. The artisan of ordinary skill will be able to readily optimize a particular solid formulation for a given application using methods well known to those of ordinary skill in the art.

The composition may comprise gel-encapsulated compounds derived from the Burkholderia strain set forth above. Such gel-encapsulated materials can be prepared by mixing a gel-forming agent (e.g., gelatin, cellulose, or lignin) with a solution of algicidal compounds and inducing gel formation of the agent.

The composition may additionally comprise a surfactant to be used for the purpose of emulsification, dispersion, wetting, spreading, integration, disintegration control, stabilization of active ingredients, and improvement of fluidity or rust inhibition. In a particular embodiment, the surfactant is a non-phytotoxic non-ionic surfactant which preferably belongs to EPA List 4B. In another particular embodiment, the nonionic surfactant is polyoxyethylene (20) monolaurate. The concentration of surfactants may range between 0.1-35% of the total formulation, preferred range is 5-25%. The choice of dispersing and emulsifying agents, such as non-ionic, anionic, amphoteric and cationic dispersing and emulsifying agents, and the amount employed is determined by the nature of the composition and the ability of the agent to facilitate the dispersion of these compositions.

In order to provide compositions containing the active ingredient(s) set forth above in the form of dusts, granules, water dispersible powders, aqueous dispersions, or emulsions and dispersions in organic liquids, the carrier or diluent agent in such compositions may be a finely divided solid, an organic liquid, water, a wetting agent, a dispersing agent, humidifying agent, or emulsifying agent, or any suitable combination of these. Generally, when liquids and wettable powders are prepared, a conditioning agent comprising one or more surface-active agents or surfactants is present in amounts sufficient to render a given composition containing the active material, the microorganism, dispersible in water or oil.

Since these compositions can be applied as a spray utilizing a liquid carrier, it is contemplated that a wide variety of liquid carriers such as, for example, water, organic solvents, decane, dodecane, oils, vegetable oil, mineral oil, alcohol, glycol, polyethylene glycol, agents that result in a differential distribution of pathogenic bacterium in water being treated. Combinations thereof and other known to artisan of ordinary skill can be used.

The present compositions can also include other substances which are not detrimental to the active ingredient(s) such as adjuvants, surfactants, binders, stabilizers and the like, which are commonly used in algicides, either singly or in combination as needed.

It will be understood by the artisan of ordinary skill that various additives or agents that predispose pests susceptible to the active ingredient set forth above are added to enhance its pesticidal action. By the phrase “additive that enhances the pesticidal action of the active ingredient” is meant any compound, solvent, reagent, substance, or agent that increases the effect of the active ingredient toward pests and more particularly, mites as compared to the pesticidal effect of the active ingredient in the absence of said additive. In some embodiments, these additives will increase the susceptibility of a particular pest to the active ingredient. Additional additives include but are not limited to agents which weaken the biological defenses of susceptible pests. Such agents can include salts, such as NaCl and CaCl2.

The composition may further comprise another microorganism and/or pesticide (e.g, nematocide, fungicide, insecticide, herbicide, algicide, aracicide). The microorganism may include but is not limited to an agent derived from Bacillus sp., Pseudomonas sp., Brevabacillus sp., Lecanicillium sp., non-Ampelomyces sp., Pseudozyma sp., Streptomyces sp, Burkholderia sp, Trichoderma sp, Gliocladium sp. Alternatively, the agent may be a natural oil or oil-product having fungicidal, herbicidal, aracidal, algicidal, nematocidal and/or insecticidal activity (e.g., paraffinic oil, tea tree oil, lemongrass oil, clove oil, cinnamon oil, citrus oil, rosemary oil).

The composition, in particular, may further comprise an insecticide. The insecticide may include but is not limited to avermectin, Bacillus thuringiensis, neem oil and azadiractin, spinosads, Chromobacterium subtsugae, eucalyptus extract, entomopathogenic bacterium or fungi such a Beauveria bassiana, and Metarrhizium anisopliae and chemical insecticides including but not limited to organochlorine compounds, organophosphorous compounds, carbamates, pyrethroids, and neonicotinoids.

The composition my further comprise a nematocide. The nematocide may include, but is not limited to chemical nematocides such as fenamiphos, aldicarb, oxamyl, carbofuran, natural product neamticide, avermectin, the fungi Paecilomyces lilacinas and Muscodor spp., the bacteria Bacillus firmus and other Bacillus spp. and Pasteuria penetrans.

The composition may further comprise a biofungicide such as extract of R. sachalinensis (Regalia) or a fungicide. Such fungicides include, but are not limited to, a single site anti-fungal agent which may include but is not limited to benzimidazole, a demethylation inhibitor (DMI) (e.g., imidazole, piperazine, pyrimidine, triazole), morpholine, hydroxypyrimidine, anilinopyrimidine, phosphorothiolate, quinone outside inhibitor, quinoline, dicarboximide, carboximide, phenylamide, anilinopyrimidine, phenylpyrrole, aromatic hydrocarbon, cinnamic acid, hydroxyanilide, antibiotic, polyamine, calamine, phthalimide, benzenoid (xylylalanine). In yet a further embodiment, the antifungal agent is a demethylation inhibitor selected from the group consisting of imidazole (e.g., triflumizole), piperazine, pyrimidine and triazole (e.g., bitertanol, myclobutanil, penconazole, propiconazole, triadimefon, bromuconazole, cyproconazole, diniconazole, fenbuconazole, hexaconazole, tebuconazole, tetraconazole, propiconazole).

The antimicrobial agent may also be a multi-site non-inorganic, chemical fungicide selected from the group consisting of a nitrile (e.g., chloronitrile or fludioxonil), quinoxaline, sulphamide, phosphonate, phosphite, dithiocarbamate, chloralkythios, phenylpyridin-amine, cyano-acetamide oxime.

The compositions may be applied using methods known in the art. Specifically, these compositions may be applied to plants or plant parts. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and genetic engineering methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds.

Treatment of the plants and plant parts with the compositions set forth above may be carried out directly or by allowing the compositions to act on their surroundings, habitat or storage space by, for example, immersion, spraying, evaporation, fogging, scattering, painting on, injecting. In the case that the composition is applied to a seed, the composition may be applied to the seed as one or more coats prior to planting the seed using one or more coats using methods known in the art.

As noted above, the compositions may be herbicidal compositions. The composition may further comprise one or more herbicides. These may include, but are not limited to, a bioherbicide and/or a chemical herbicide. The bioherbicide may be selected from the group consisting of clove oil, cinnamon oil, lemongrass oil, citrus oil, orange peel oil, tentoxin, cornexistin, AAL-toxin, manuka oil, leptospermone, thaxtomin, sarmentine, momilactone B, sorgoleone, ascaulatoxin and ascaulatoxin aglycone. The chemical herbicide may include, but is not limited to, diflufenzopyr and salts thereof, dicamba and salts thereof, topramezone, tembotrione, S-metolachlor, atrazine, mesotrione, primisulfuron-methyl, 2,4-dichlorophenoxyacetic acid, nicosulfuron, thifensulfuron-methyl, asulam, metribuzin, diclofop-methyl, fluazifop, fenoxaprop-p-ethyl, asulam, oxyfluorfen, rimsulfuron, mecoprop, and quinclorac, thiobencarb, clomazone, cyhalofop, propanil, bensulfuron-methyl, penoxsulam, triclopyr, imazethapyr, halosulfuron-methyl, pendimethalin, bispyribac-sodium, carfentrazone ethyl, sodium bentazon/sodium acifluorfen, glyphosate, glufosinate and orthosulfamuron.

Herbicidal compositions may be applied in liquid or solid form as pre-emergence or post-emergence formulations.

For pre-emergence dry formulations, the granule size of the carrier is typically 1-2 mm (diameter) but the granules can be either smaller or larger depending on the required ground coverage. Granules may comprise porous or non-porous particles.

For post-emergence formulations, the formulation components used may contain smectite clays, attapulgite clays and similar swelling clays, thickeners such as xanthan gums, gum Arabic and other polysaccharide thickeners as well as dispersion stabilizers such as nonionic surfactants (for example polyoxyethylene (20) monolaurate).

In a particular embodiment, the composition may comprise in addition to the active ingredient another microorganism and/or algicide and/or acaricide. The microorganism may include but is not limited to an agent derived from Bacillus sp., Brevibacillus sp., and Streptomyces sp.

The compositions may also as set forth above, be algicidal compositions which can further comprise other algicides such as copper sulphate, diquat or thaxtomin A.

The compositions may be acaricidal compositions which can further comprise other acaricides such as antibiotics, carbamates, formamidine acaricides, pyrethroids, mite growth regulators, organophosphate acaricides and diatomaceous earth.

The compositions and pesticidal compounds derived from the Burkholderia strain set forth herein may be used as pesticides, particularly as insecticides, nematocides, fungicides, algicides, acaricides and herbicides.

Specifically, nematodes that may be controlled using the method set forth above include but are not limited to parasitic nematodes such as root-knot, ring, sting, lance, cyst, and lesion nematodes, including but not limited to free living nematodes, Meloidogyne, Heterodera and Globodera spp; particularly Meloidogyne incognita (root knot nematodes), as well as Globodera rostochiensis and globodera pailida (potato cyst nematodes); Heterodera glycines (soybean cyst nematode); Heterodera schachtii (beet cyst nematode); Oligonychus pratensis (Banks grass mite); Eriophyes cynodoniensis (Bermuda grass mite); Bryobia praetiosa (Clover mite)—and Heterodera avenae (cereal cyst nematode).

Phytopathogenic insects controlled by the method of the present invention include, but are not limited to, insects from the order (a) Lepidoptera, for example, Acleris spp., Adoxophyes spp., Aegeria spp., Agrotis spp., Alabama argillaceae, Amylois spp., Anticarsia gemmatalis, Archips spp., Argyrotaenia spp., Autographa spp., Busseola fusca, Cadra cautella, Carposina nipponensis, Chilo spp., Choristoneura spp., Clysia ambiguella, Cnaphalocrocis spp., Cnephasia spp., Cochylis spp., Coleophora spp., Crocidolomia binotalis, Cryptophlebia leucotreta, Cydia spp., Diatraea spp., Diparopsis castanea, Earias spp., Ephestia spp., Eucosma spp., Eupoecilia ambiguella, Euproctis spp., Euxoa spp., Grapholita spp., Hedya nubiferana, Heliothis spp., Hellula undalis, Hyphantria cunea, Keiferia lycopersicella, Leucoptera scitella, Lithocollethis spp., Lobesia botrana, Lymantria spp., Lyonetia spp., Malacosoma spp., Mamestra brassicae, Manduca sexta, Operophtera spp., Ostrinia nubilalis, Pammene spp., Pandemis spp., Panolis flammea, Pectinophora gossypiella, Phthorimaea operculella, Pieris rapae, Pieris spp., Plutella xylostella, Prays spp., Scirpophaga spp., Sesamia spp., Sparganothis spp., Spodoptera spp., Synanthedon spp., Thaumetopoea spp., Tortrix spp., Trichoplusia ni and Yponomeuta spp.; (b) Coleoptera, for example, Agriotes spp., Anthonomus spp., Atomaria linearis, Chaetocnema tibialis, Cosmopolites spp., Curculio spp., Dermestes spp., Diabrotica spp., Epilachna spp., Eremnus spp., Leptinotarsa decemlineata, Lissorhoptrus spp., Melolontha spp., Orycaephilus spp., Otiorhynchus spp., Phlyctinus spp., Popillia spp., Psylliodes spp., Rhizopertha spp., Scarabeidae, Sitophilus spp., Sitotroga spp., Tenebrio spp., Tribolium spp. and Trogoderma spp.; (c) Orthoptera, for example, Blatta spp., Blattella spp., Gryllotalpa spp., Leucophaea maderae, Locusta spp., Periplaneta spp. and Schistocerca spp.; (d) Isoptera, for example, Reticulitermes spp.; (e) Psocoptera, for example, Liposcelis spp.; (f) Anoplura, for example, Haematopinus spp., Linognathus spp., Pediculus spp., Pemphigus spp. and Phylloxera spp.; (g) Mallophaga, for example, Damalinea spp. and Trichodectes spp.; (h) Thysanoptera, for example, Frankliniella spp., Hercinotnrips spp., Taeniothrips spp., Thrips palmi, Thrips tabaci and Scirtothrips aurantii; (i) Heteroptera, for example, Cimex spp., Distantiella theobroma, Dysdercus spp., Euchistus spp., Eurygaster spp., Leptocorisa spp., Nezara spp., Piesma spp., Rhodnius spp., Sahlbergella singularis, Scotinophara spp., Oncopeltus spp. Lygys spp. and Tniatoma spp.; (j) Homoptera, for example, Aleurothrixus floccosus, Aleyrodes brassicae, Aonidiella spp., Aphididae, Aphis spp., Aspidiotus spp., Bemisia tabaci, Ceroplaster spp., Chrysomphalus aonidium, Chrysomphalus dictyospermi, Coccus hesperidum, Empoasca spp., Eriosoma larigerum, Erythroneura spp., Gascardia spp., Laodelphax spp., Lecanium corni, Lepidosaphes spp., Macrosiphus spp., Myzus spp., Nephotettix spp., Nilaparvata spp., Paratoria spp., Pemphigus spp., Planococcus spp., Pseudaulacaspis spp., Pseudococcus spp., Psylla spp., Pulvinaria aethiopica, Quadraspidiotus spp., Rhopalosiphum spp., Saissetia spp., Scaphoideus spp., Schizaphis spp., Sitobion spp., Trialeurodes vaporariorum, Trioza erytreae and Unaspis citri; (k) Hymenoptera, for example, Acromyrmex, Atta spp., Cephus spp., Diprion spp., Diprionidae, Gilpinia polytoma, Hoplocampa spp., Lasius spp., Monomorium pharaonis, Neodiprion spp., Solenopsis spp. and Vespa spp.; (1) Diptera, for example, Aedes spp., Antherigona soccata, Bibio hortulanus, Calliphora erythrocephala, Ceratitis spp., Chrysomyia spp., Culex spp., Cuterebra spp., Dacus spp., Drosophila melanogaster, Fannia spp., Gastrophilus spp., Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis pomonella, Sciara spp., Stomoxys spp., Tabanus spp., Tannia spp. and Tipula spp.; (m) Siphonaptera, for example, Ceratophyllus spp. and Xenopsylla cheopis and (n) from the order Thysanura, for example, Lepisma saccharina. The active ingredients according to the invention may further be used for controlling crucifer flea beetles (Phyllotreta spp.), root maggots (Delia spp.), cabbage seedpod weevil (Ceutorhynchus spp.) and aphids in oil seed crops such as canola (rape), mustard seed, and hybrids thereof, and also rice and maize. In a particular embodiment, the insect may be a member of the Spodoptera, more particularly, Spodoptera exigua, Myzus persicae, Plutella xylostella or Euschistus sp.

The substances and compositions may also be used to modulate emergence in either a pre-emergent or post-emergent formulation of monocotyledonous, including sedges and grasses, or dicotyledonous weeds. In a particular embodiment, the weeds may include, but not be limited to, Chenopodium sp. (e.g. C. album), Abutilon sp. (e.g. A. theophrasti), Helianthus sp. (e.g. H. annuus), Ludwigia sp. (e.g. L. hexapetala), Ambrosia sp. (e.g. A. artemesifolia), Amaranthus sp. (e.g., A. retroflexus, A. palmeri), Convolvulus sp. (e.g. C. arvensis), Ipomoeae sp., Brassica sp. (e.g. B. kaber), Raphanus sp., Taraxacum sp. (e.g. T. officinale), Centaurea sp. (e.g. C. solstitalis), Conyza sp. (e.g. C. bonariensis), Cirsium sp. (e.g. C. arvense), Lepidium sp., Gallium sp., Solanum sp. (e.g. S. nigrum), Malva sp. (e.g. M. neglecta), Cyperus sp. (e.g. C. rotundus), Oxalis sp., Euphorbia sp., Trifolium sp., Medicago sp., Hydrilla sp., Azolla sp., Digitaria sp. (e.g. D. sanguinalis), Setaria sp. (e.g. S. lutescens), Cynodon dactylon, Bromus sp. (e.g., B. tectorum), Poa sp. (e.g. P. annua, P. pratensis), Lollium sp. (e.g, L. perenne), Sorghum sp. (e.g. S. halepense), Arundo donax, Festuca sp. (e.g. F. arundinaceae), Echinochloa sp. (e.g., E. crus-galli, E. phyllopogon).

The Burkholderia strain, compounds and compositions set forth above may also be used as a fungicide. The targeted fungus may be a Fusarium sp., Botrytis sp., Monilinia sp., Colletotrichum sp, Verticillium sp.; Microphomina sp., Phytophtora sp, Mucor sp., Podosphaera sp., Rhizoctonia sp., Peronospora sp., Geotrichum sp., Phoma, and Penicillium. In another most particular embodiment, the bacteria are Xanthomonas.

The substance or compositions can be used to control, reduce and or eliminate the growth and proliferation of marine and non-marine micro and macro algae including but not limited to unicellular, multicellular and diatom, red-, green- and bluegreen-algae such as Pseudokirchneriella subcapitata, Rhizoclonium sp., Cladophoera sp., Anabaena sp., Nostoc sp., Hydrodictyon sp., Chara sp, Microcystis and Didymo sp., Chlamydomonas sp., Scenedesmus sp., Oscillatoria sp., Volvox sp., Navicula sp, Oedogonium sp., Spirogyra sp., Batrichospermum sp., Rhodymenia sp., Callithamnion sp., Undaria sp., through algaecide and algaestatic activity.

The active ingredient(s) and compositions set forth above may be applied to locations containing algae. These include but are not limited to a body of water such as a pond, lake, stream, river, aquarium, water treatment facility, power plant or a solid surface, such as plastic, concrete, wood, fiberglass, pipes made of iron and polyvinyl choride, surfaces covered wih coating materials and/or paints.

As noted above, the active ingredient(s) and compositions set forth above may be applied to locations containing arachnids, such as mites, including but not limited to, Panonychus sp. such as Panonychus citri (citrus red mite), and Panonychus ulmi (red spider mite), Tetranychus sp. such as Tetranychus kanzawi (Kanzawa spider mite), Tetranychus urticae (2 spotted spider mite), Tetranychus pacificus (Pacific spider mite), Tetranychus turkestanii (Strawberry mite) and Tetranychus cinnabarinus (Carmine spider mite), Oligonychus sp. such as Oligonychus panicae (avacado brown mite), Oligonychus perseae (persea mite), Oligonychus pratensis (Banks grass mite) and Oligonychus coffeae, Aculus sp. such as Aculus cornatus (Peach silver mite), Aculus fockeni (plum rust mite) and Aculus lycopersici (tomato russet mite), Eotetranychus sp. such as Eotetranychus wilametti, Eotetranychus yumensis (yuma spider mite) and Eotetranychus sexmaculatis (6-spotted mite), Bryobia rubrioculus (brown mite), Epitrimerus pyri (pear rust mite), Phytoptus pyri (Pear leaf blister mite), Acalitis essigi (red berry mite), Polyphagotarsonemus latus (Broad mite), Eriophyes sheldoni (citrus bud mite), Brevipalpus lewisi (citrus flat mite), Phylocoptruta oleivora (citrus rust mite), Petrobia lateens (Brown wheat mite), Oxyenus maxwelli (olive mite), Rhizoglyphus spp., Tyrophagus spp., Diptacus gigantorhyncus (bigheaded plum mite) and Penthaleaa major (winter grain mite), Avocado red mite, Flat mite, black and red Mango spider mite, Papaya leaf edgeroller mite, Texas citrus mite, European red mite, Grape erineum mite (blister mite), Pacific spider mite, Willamette spider mitePink citrus rust mite.

Such locations may include but are not limited to crops that are infested with such mites or other arachnids (e.g., aphenids).

The invention will now be described in greater detail by reference to the following non-limiting examples.

The compositions and methods set forth above will be further illustrated in the following, non-limiting Examples. The examples are illustrative of various embodiments only and do not limit the claimed invention regarding the materials, conditions, weight ratios, process parameters and the like recited herein.

Isolation and identification of the microbe. Isolation of the microorganism. The microbe is isolated using established techniques know to the art from a soil sample collected under an evergreen tree at the Rinnoji Temple, Nikko, Japan. The isolation is done using potato dextrose agar (PDA) using a procedure described in detail by Lorch et al. , 1995. In this procedure, the soil sample is first diluted in sterile water, after which it is plated in a solid agar medium such as potato dextrose agar (PDA). The plates are grown at 25° C. for five days, after which individual microbial colonies are isolated into separate PDA plates. The isolated bacterium is gram negative, and it forms round, opaque cream-colored colonies that change to pink and pinkish-brown in color and mucoid or slimy over time.

Identification on the microorganism. The microbe is identified based on gene sequencing using universal bacterial primers to amplify the 16S rRNA region. The following protocol is used: Burkholderia sp. A396 is cultured on potato-dextrose agar plates. Growth from a 24 hour-old plate is scraped with a sterile loop and re-suspended in DNA extraction buffer. DNA is extracted using the MoBio Ultra Clean Microbial DNA extraction kit. DNA extract is checked for quality/quantity by running 5 μl on a 1% agarose gel.

PCR reactions are set up as follows: 2 μl DNA extract, 5 μl PCR buffer, 1 μl dNTPs (10 mM each), 1.25 μl forward primer (27F; (SEQ ID NO:1), 1.25 μl reverse primer (907R; (SEQ ID NO:2)) and 0.25 μl Taq enzyme. The reaction volume is made up to 50 μl using sterile nuclease-free water. The PCR reaction includes an initial denaturation step at 95° C. for 10 minutes, followed by 30 cycles of 94° C./30 sec, 57° C./20 sec, 72° C./30 sec, and a final extension step at 72° C. for 10 minutes.

The product's approximate concentration and size is calculated by running a 5 μl volume on a 1% agarose gel and comparing the product band to a mass ladder.

Excess primers, dNTPs and enzyme are removed from the PCR product with the MoBio PCR clean up kit. The cleaned PCR product as directly sequenced using primers 27F (same as above), 530F (SEQ ID NO:3)), 1114F (SEQ ID NO:4)) and 1525R (SEQ ID NO:5)), 1100R (SEQ ID NO:6)), 519R (SEQ ID NO:7).

The 16S rRNA gene sequence of strain A396 is compared with the available 16S rRNA gene sequences of representatives of the β-proteobacteria using BLAST. Strain A395 A396 is closely related to members of the Burkholderia cepacia complex, with 99% or higher similarity to several isolates of Burkholderia multivorans, Burkholderia vietnamensis, and Burkholderia cepacia. A BLAST search excluding the B. cepacia complex, showed 98% similarity to B. plantarii, B. gladioli and Burkholderia sp. isolates.

A distance tree of results using the neighbor joining method, showed that A396 is related to Burkholderia multivorans and other Burkholderia cepacia complex isolates. Burkholderia plantarii and Burkholderia glumae grouped in a separate branch of the tree.

The isolated Burkholderia strain was found to contain the following sequences: Forward sequence, DNA sequence with 27F primer, 815 nucleotides (SEQ ID NO:8); Reverse sequence, 1453 bp, using primers 1525R, 1100R, 519R (SEQ ID NO:9); Reverse sequence 824 bp using primer 907R (SEQ ID NO: 10); Forward sequence 1152 bp using primer 530F (SEQ ID NO:11); Forward sequence 1067 bp using 1114F primer (SEQ ID NO:12); Reverse sequence 1223 bp using 1525R primer (SEQ NO:13); Reverse sequence 1216 bp using 1100R primer (SEQ ID NO:14); Reverse sequence 1194 bp using 519R primer (SEQ ID NO:15).

Proof that Burkholderia A396 does not belong to Burkholderia cepacia complex. Molecular Biology work using specific PCR primers. In order to confirm the identification of Burkholderia A396 as Burkholderia multivorans, additional sequencing of housekeeping genes is performed. Burkholderia multivorans is a known member of the Burkholderia cepacia complex. Efforts are focused on PCR of recA genes, as described by Mahenthiralingam et al., 2000. The following primers are used: (a) BCR1 and BCR2 set forth in Mahenthiralingam et al., 2000 to confirm B. cepacia complex match and (b) BCRBM1 and BCRBM2 set forth Mahenthiralingam et al, 2000 to confirm B. multivorans match. A product-yielding PCR reaction for the first primer set would confirm that the microbe belongs to the B. cepacia complex. A product-yielding PCR reaction for the second primer set would confirm that the microbe is indeed B. multivorans.

No PCR product is obtained for either pair of primers. The performance of the PCR reaction and primers is tested using Burkholderia multivorans ATCC 17616 (positive control) and Pseudomonas fluorescens (negative control). Strong bands are observed both for B. multivorans using both sets of primers. No bands are observed for Pseudomonas fluorescens. The results indicate that A396 is a Burkholderia, but not a member of the B. cepacia complex, and not Burkholderia multivorans. This is also demonstrated in a comparative culture experiment in which both A396 and a type culture of B. multivorans are grown side-by-side in a shake culture, and the growth is monitored daily using optical density measurements at 600 nm. Under the set conditions, species A396 grew much faster than the B. multivorans type strain (FIG. 1).

DNA-DNA Hybridization. In order to confirm that isolate A396 is a new species of Burkholderia, a DNA-DNA hybridization experiment with Burkholderia multivorans (the closest 16SrRNA sequence match) is conducted. Biomass for both A396 and B. multivorans is produced in ISP2 broth, grown over 48 hours at 200 rpm/25° C. in Fernbach flasks. The biomass is aseptically harvested by centrifugation. The broth is decanted and the cell pellet is resuspended in a 1:1 solution of water:isopropanol. DNA-DNA hybridization experiments are performed by the DSMZ, the German Collection of Microorganisms and Cell Cultures in Germany. DNA is isolated using a French pressure cell (Thermo Spectronic) and is purified by chromatography on hydroxyapatite as described by Cashion et al., 1977. DNA-DNA hybridization is carried out as described by De Ley et al., 1970 under consideration of the modifications described by Huss et al., 1983 using a model Cary 100 Bio UV/VIS-spectrophotometer equipped with a Peltier thermostatted 6×6 multicell changer and a temperature controller with in-situ temperature probe (Varian). DSMZ reported % DNA-DNA similarly between A396 and Burkholderia multivorans of 37.4%. The results indicate that Burkholderia sp strain A396 does not belong to the species Burkholderia multivorans when the recommendations of a threshold value of 70% DNA-DNA similarity for the definition of bacterial species by the ad hoc committee (Wayne et al., 1987) are considered.

Biochemical profile using Biolog GN2 plates. For the carbon source utilization profile, A396 is grown overnight on Potato Dextrose Agar (PDA). The culture is transferred to BUG agar to produce an adequate culture for Biolog experiments as recommended by the manufacturer (Biolog, Hayward, Calif.).

The biochemical profile of the microorganism is determined by inoculating onto a Biolog GN2 plate and reading the plate after a 24-hour incubation using the MicroLog 4-automated microstation system. Identification of the unknown bacteria is attempted by comparing its carbon utilization pattern with the Microlog 4 Gram negative database.

No clear definitive matches are found to the Biolog profile. The closest matches all had less than 35% similarity with A396: Pseudomonas spinosa (Burkholderia), Burkholderia cepacia, and Burkholderia pseudomallei. The results are shown in Table 1.

TABLE 1
Biochemical Profile of A396

| Substrate | Result | Substrate | Result |
|---|---|---|---|
| Cyclodextrin | − | L-arabinose | − |
| Dextrin | − | D-arabitol | − |
| Glycogen | − | D-cellobiose | − |
| Tween 40 | + | Erythritol | − |
| Tween 80 | + | D-Fructose | − |
| N-acetyl-D-Galactoseamine | − | L-Fucose | − |
| N-acetyl-D-glucosamine | − | D-Galactose | +/− |
| Adonitol | − | Gentibiose | − |
| Succinic Acid Mon-methyl | − | D-Glucose | + |
| ester | | | |
| Acetic acid | − | m-Inositol | − |
| Cis-aconitic acid | − | D-Lactose | − |
| Citric acid | − | Lactulose | − |
| Formic acid | + | Maltose | − |
| D-Galactonic Acid Lactone | − | D-Mannitol | − |
| D-Galacturonic Acid | − | D-Mannose | − |
| D-Gluconic acid | − | D-Melibiose | − |
| D-Glucosaminic acid | − | β-methyl-D-glucoside | − |
| D-Glucuronic Acid | − | D-Psicose | − |
| α-hydroxyburytic acid | − | D-Raffinose | − |
| β-hydroxybutyric acid | + | L-Rhamonose | − |
| γ-hydroxybutyric acid | − | D-Sorbitol | − |
| p-hydroxyphenylacetic acid | − | Sucrose | − |
| Itaconic acid | − | D-Trehalose | + |
| α-keto butyric acid | − | Turanose | − |
| α-keto glutaric acid | − | Xylitol | − |
| α-ket valeric acid | − | Pyruvic Acid Methyl | − |
| | | esther | |
| D,L-Lactic acid | − | Uridine | − |
| Malonic acid | − | Thymidine | − |
| Propionic acid | + | Phenyethyl-amine | − |
| Quinic acid | − | Putrescine | − |
| D-Saccharic acid | − | 2-aminoethanol | − |
| Sebacic acid | − | 2,3-Butanediol | − |
| Succinic Acid | + | Glycerol | +/− |
| Bromosuccinic acid | − | D,L-a-glycerol phosphate | +/− |
| Succinamic acid | − | α-D-Glucose-1- | − |
| | | phosphate | |
| Glucuronamide | − | D-glucose-6-phosphate | + |
| L-alaninamide | + | γ-amino butyric acid | + |
| D-Alanine | − | Urocanic acid | − |
| L-alanine | + | Inosine | − |
| L-alanyl-glycine | − | L-phenylalanine | + |
| L-asparagine | + | L-proline | − |
| L-aspartic acid | +/− | L-pyroglutamic acid | − |
| L-glutamic acid | + | D-serine | − |
| Glycyl-L-Aspartic acid | − | L-serine | − |
| Glycyl-L-glutamic acid | − | L-threonine | − |
| L-histidine | − | D,L-carnitine | − |
| Hydroxy-L-proline | + | L-ornithine | − |

| | |
|---|---|
| L-leucine | − |

Fatty acid composition. After incubation for 24 hours at 28° C., a loopful of well-grown cells are harvested and fatty acid methyl esters are prepared, separated and identified using the Sherlock Microbial Identification System (MIDI) as described (see Vandamme et al., 1992). The predominant fatty acids present in the Burkholderia A396 are as follows: 16:0 (24.4%), cyclo 17:0 (7.1%), 16:0 3-OH (4.4%), 14:0 (3.6%), 19:0 ω8c (2.6%) cyclo, 18:0 (1.0%). Summed feature 8 (comprising 18:1 ω7c) and summed feature 3 (comprising of 16:1 ω7c and 16:1 ω6c) corresponded to 26.2% and 20.2% of the total peak area, respectively. Summed feature 2 comprising 12:0 ALDE, 16:1 iso I, and 14:0 3-OH) corresponded to 5.8% of the total peak area while summed feature 5 comprising 18:0 ANTE and 18:2 ω6,9c corresponded to 0.4%. Other fatty acids detected in A396 in minor quantities included: 13:1 at 12-13 (0.2%), 14:1 ω5c (0.2%), 15:0 3-OH (0.13%), 17:1 ω7c (0.14%), 17:0 (0.15%), 16:0 iso 3-OH (0.2%), 16:0 2-OH (0.8%), 18:1 ω7c 11-methyl (0.15%), and 18:1 2-OH (0.4%).

A comparison of the fatty acid composition of A396 with those of known microbial strains in the MIDI database suggested that the fatty acids in the novel strain A396 were most similar with those of Burkholderia cenocepacia.

Resistance to Antibiotics. Antibiotic susceptibility of Burkholderia A396 is tested using antibiotic disks on Muller-Hinton medium as described in PML Microbiological's technical data sheet #535. Results obtained after 72-hour incubation at 25oC are presented in Table 2 below.

TABLE 2
Susceptibility of MBI-206 to various antibiotics.

| | Concentration (μg) | Susceptible |
|---|---|---|
| Tetracycline | 30 | − |
| Kanamycin | 30 | +++ |
| Erythromycin | 15 | − |
| Streptomycin | 10 | − |
| Penicillin | 10 | − |
| Ampicillin | 10 | − |
| Oxytetracycline | 30 | − |
| Chloramphenicol | 30 | ++ |
| Ciprofloxacin | 5 | ++ |
| Gentamicin | 10 | − |
| Piperacillin | 100 | +++ |
| Cefuroxime | 30 | − |
| Imipenem | 10 | +++ |
| Sulphamethoxazole- | 23.75/25 | ++ |
| Trimethoprim | | |

+++ very susceptible,
++ susceptible,
− resistant

The results indicate that the antibiotic susceptibility spectrum of Burkholderia A396 is quite different from pathogenic B. cepacia complex strains. Burkholderia A396 is susceptible to kanamycin, chloramphenicol, ciprofloxacin, piperacillin, imipenem, and a combination of sulphamethoxazole and trimethoprim. As a comparison, Zhou et al., 2007 tested the susceptibility of 2,621 different strains in B. cepacia complex isolated from cystic fibrosis patients, and found that only 7% and 5% of all strains were susceptible to imipenem or ciprofloxacin, respectively. They also found 85% of all strains to be resistant to chloramphenicol (15% susceptible), and 95% to be resistant (5% susceptible) to the combination of sulphamethoxazole and trimethoprim. Results of Zhou et al., 2007 are similar to those of Pitt et al., 1996 who determined antibiotic resistance among 366 B. cepacia isolates and reported that most of them are resistant to ciprofloxacin, cefuroxime, imipenem, chloramphenicol, tetracycline, and sulphametoxacole.

Example 2: Burkholderia formulation and isolation of Fractions from Formulated Product. The following procedure is used for the purification of compounds extracted from a formulated product of MBI-206 containing a whole cell broth of a culture of Burkholderia sp.:

The culture broth derived from the 10-L fermentation Burkholderia (A396) in Hy soy growth medium and formulated using methyl 0.1% and propyl paraben, 0.1% hexanol 0.67% and Glycosperse 0-20, 0.67% is extracted with Amberlite XAD-7 resin (Asolkar et al., “Weakly cytotoxic polyketides from a marine-derived Actinomycete of the genus Streptomyces strain CNQ-085.” J. Nat. Prod. 69:1756-1759. 2006) by shaking the cell suspension with resin at 225 rpm for two hours at room temperature. The resin and cell mass are collected by filtration through cheesecloth and washed with DI water to remove salts. The resin, cell mass, and cheesecloth are then soaked for 2 h in acetone after which the acetone is filtered and dried under vacuum using rotary evaporator to give the crude extract (MBI-206-FP-CE). The crude extract is then fractionated by using reversed-phase C18 vacuum liquid chromatography (H2O/CH3OH; gradient 80:20 to 0:100%) to give 10 fractions (see FIG. 1 for schematic). These fractions are then concentrated to dryness using rotary evaporator and the resulting dry residues are screened for biological activity using a whole plant herbicidal assay. The active fractions, fractions 3, 4, 5 and 6 and indicated as MBI-206-FP-3, MBI-206-FP-4, MBI-206-FP-5, and MBI-206-FP-6 respectively are then subjected to repeatedly to reversed phase HPLC separation (Spectra System P4000 (Thermo Scientific) to give pure compounds, which are then screened in above-mentioned bioassays to locate/identify the active compounds (see FIG. 2).

Analysis of Formulation fractions. These fractions are analyzed on a Thermo high performance liquid chromatography (HPLC) instrument equipped with Finnigan Surveyor PDA plus detector, autosampler plus, MS pump and a 4.6 mm×100 mm Luna C18 5 μm column (Phenomenex). The solvent system consisted of water (solvent A) and acetonitrile (solvent B). The mobile phase begins at 10% solvent B and is linearly increased to 100% solvent B over 20 min and then kept for 4 min, and finally returned to 10% solvent B over 3 min and kept for 3 min. The flow rate is 0.5 mL/min. The injection volume is 10 μL and the samples are kept at room temperature in an auto sampler.

To discover the identity of the compound, additional spectroscopic data such as LC/MS and UV are recorded. Compound corresponding to fraction 5, with a retention time of 17.45 minutes is not found in any of the starting materials, which indicates that the compound is a product of a chemical reaction between natural products in the microbial fermentation broth and one or more of the compounds found in the formulation agents. Specifically, this fraction was analyzed using ESI-LCMS on a Thermo Finnigan LCQ Deca XP Plus electrospray (ESI) instrument using both positive and negative ionization modes in a full scan mode (m/z 100-1500 Da) on a LCQ DECA XPplus Mass Spectrometer (Thermo Electron Corp., San Jose, Calif.). Mass spectroscopy analysis is performed under the following conditions: The flow rate of the nitrogen gas was fixed at 30 and 15 arb for the sheath and aux/sweep gas flow rate, respectively. Electrospray ionization was performed with a spray voltage set at 5000 V and a capillary voltage at 35.0 V. The capillary temperature was set at 400° C. The data was analyzed on Xcalibur software. The additional new compounds found in fraction 5 were found to have a molecular weight (MW) of 194 (RT=14.74 min) and 222 (RT=17.43 min).

Bioassay. Healthy radish plants with two to three true leaves were selected for testing. The radish plants are 13 days old at treatment. The plants are sorted so that all treatments are equivalent in foliage surface area and plant height. The pots are labeled with treatment number and repetition number. Three repetitions per treatment are tested.

Ten fractions of MBI-206 formulated product are tested. The fractions are at a concentration of 10 mg/ml. The crude extracts of the formulated product and broth are also tested. An untreated control (treated with deionized water) and a positive control (RoundUp Super Concentrate at a rate of 2.5 fluid ounces per gallon) are included in the test.

The following treatments were tested as shown in Table 3:

TABLE 3
Test Description

| Treatment | Sample ID | Description |
|---|---|---|
| 1 | MBI-206-FP-F1 | 4% ethanol/water (0.2% Glycosperse) |
| 2 | MBI-206-FP-F2 | 4% ethanol/water (0.2% Glycosperse) |
| 3 | MBI-206-FP-F3 | 4% ethanol/water (0.2% Glycosperse) |
| 4 | MBI-206-FP-F4 | 4% ethanol/water (0.2% Glycosperse) |
| 5 | MBI-206-FP-F5 | 4% ethanol/water (0.2% Glycosperse) |
| 6 | MBI-206-FP-F6 | 4% ethanol/water (0.2% Glycosperse) |
| 7 | MBI-206-FP-F7 | 4% ethanol/water (0.2% Glycosperse) |
| 8 | MBI-206-FP-F8 | 4% ethanol/water (0.2% Glycosperse) |
| 9 | MBI-206-FP-F9 | 4% ethanol/water (0.2% Glycosperse) |
| 10 | MBI-206-FP-F10 | 4% ethanol/water (0.2% Glycosperse) |
| 11 | MBI-206-FP-CE | 4% ethanol/water (0.2% Glycosperse) |
| 12 | MBI-206-CE (broth) | 4% ethanol/water (0.2% Glycosperse) |
| 13 | UTC | UTC (DI water) |
| 14 | Positive Control | Pos. Control (RoundUp @ 2.5 fl oz/gal |
| | | (AI: glyphosate @ 50.2%) |

All products and treatments are well shaken prior to application. Treatments are applied using a nozzle from a 2-ounce spray bottle. Separate spray nozzles were used for each treatment. The plant foliage is sprayed evenly and with a moderate volume (i.e. neither a light misting nor a heavy application that resulted in runoff). Two milliliters of each treatment are sprayed simultaneously over the three repetitions of each treatment so that each plant is treated with approximately 0.67 milliliters of treatment solution.

The plants are allowed to air dry and are then randomized in holding trays. Each tray is labeled with the experiment name and treatment date and placed on the laboratory greenhouse shelves. The laboratory greenhouse maintains a temperature of 70-80° F. and a relative humidity of 30-40%. Throughout the bioassay, plants are watered from below by filling the holding trays with an appropriate amount of water so that plant foliage remained dry.

Results are taken at 3, 8, and 14 days after treatment. Symptoms included foliage burning and plant stunting. The following rating scale, shown in Table 4 is used to quantify efficacy. Ratings are determined by observing the following factors relative to the plants of the untreated control: overall plant health, average plant height, and foliage health. Symptoms of affected plants may include discolored/spotted/burnt/bleached foliage, warped/twisted/curled leaves, side branching (due to damaged apical meristem), plant dieback, or death.

TABLE 4
Rating Scale

| | |
|---|---|
| 0 | −0% control symptoms |
| 0.5 | −≦5% control symptoms |
| 1 | −10% control symptoms |
| 2 | −25% control symptoms |
| 3 | −50% control symptoms |
| 4 | −75% control symptoms |
| 5 | −100% control symptoms |

The mean of three readings is shown in FIG. 2. In a whole plant herbicide test, fractions 4 and 5 show good herbicidal activity (see FIG. 2).

Isolation of Pesticidal Compounds from Formulation. This fraction was further purified using a HPLC C-18 column (Phenomenex, Luna 10u C18(2) 100 A, 250×30), water:acetonitrile gradient solvent system (0-10 min; 80% aqueous CH3CN, 10-25 min; 80-65% aqueous CH3CN, 25-50 min; 65-50% aqueous CH3CN, 50-60 min; 50-70% aqueous CH3CN, 60-80 min; 70-0% aqueous CH3CN, 80-85 min; 0-20% aqueous CH3CN) at 8 mL/min flow rate and UV detection of 210 nm, to give butyl paraben, retention time 59.15 min (MBI206-FP-F5H32) and hexyl paraben, retention time 74.59 min (MBI206-FP-F5H40) respectively.

NMR Spectroscopy Analysis of Compounds. NMR spectra were measured on a Bruker 600 MHz gradient field spectrometer. The reference is set on the internal standard tetramethylsilane (TMS, 0.00 ppm).

Structure elucidation of hexyl paraben (MBI206-FP-F5H40). The active compound was isolated as a colorless solid, with UV absorption at 248 nm. The (−) ESIMS showed molecular ion at 221 (M−H) corresponding to the molecular weight of 222. The compound exhibited 1H NMR δ signals at 7.90, 6.85, 4.28, 1.76, 1.46, 1.38, 1.37, 0.94 and has 13C NMR values of 166.84, 162.12, 131.34 (2C), 121.04, 114.83 (2C), 64.32, 31.25, 28.43, 25.45, 22.18. 12.93. The molecular formula of C13H18O3 (5 degrees of unsaturation), was assigned by combination of NMR and ESI mass spectrometry data. The 1H NMR spectrum exhibited signals for an A2B2-type aromatic signals at δ 7.90, 2H d, J=8.5 Hz, and 6.85, 2H d, J=8.5 Hz. Furthermore, the 1H NMR spectrum revealed the presence of —CH2-CH2-CH2-CH2-CH2-CH3 group, at δ 4,28, 2H, t, J=7.3 Hz; 1,76, 2H, m; 1.46, 2H, m; 1.38, 2H. m; 1.37, 2H, m, and 0.94, 3H, t, J=7.3 Hz. From an analysis of the foregoing spectral data, the structure of the aromatic polyketide was established as hexyl paraben, which was confirmed by detail analysis of the COSY, HMQC and HMBC experiments. A literature search revealed that this compound has been reported as synthetic compound.

Structure elucidation of butyl paraben (MBI206-FP-F5H32). This compound was obtained as a colorless solid with UV max at 248 nm. The LCMS analysis in the negative mode showed molecular ion at m/z 193 corresponding to the molecular formula 194. By comparison of the UV, MS and NMR data with that of hexyl paraben with MW 222, this compound was found to be the analogue of hexyl paraben. The only difference between them was only in the side chain. Thus, the structure of butyl paraben was assigned to this compound with MW 194. A search in the literature suggested that this compound is also known as a synthetic compound.

Herbicidal Activity. The pure compounds (butyl paraben [MBI206-FP-F5H32] and hexyl paraben [MBI206-FP-F5H40]) obtained from fraction 5 were tested at a concentration of 10 mg/ml. An untreated control (treated with deionized water), the formulation blank (at 3% v/v & 10% v/v), and a positive control (RoundUp Super Concentrate at a rate of 2.5 fluid ounces per gallon) are included in the test.

The following treatments were tested as shown in Table 5:

TABLE 5
Treatment Regimen
Test description

| Treat- | | | Vol (mL) |
|---|---|---|---|
| ment | Sample ID | Description | of Sample |
| 1 | UTC | UTC (DI water) | 2 |
| 2 | Butyl paraben | 4% ethanol/water | 2 |
| | (MBI206-FP- | (0.2% Glycosperse) | |
| | F5H32) | | |
| 3 | Hexyl paraben | 4% ethanol/water | 2 |
| | (MBI206-FP- | (0.2% Glycosperse) | |
| | F5H40) | | |
| 4 | Blank | Formulation Blank @ 3% v/v | 2 |
| 5 | Blank | Formulation Blank @ 10% v/v | 2 |
| 6 | Positive Control | Positive Control | 2 |
| | | (RoundUp @ 2.5 fl oz/gal | |
| | | (AI: glyphosate @ 50.2%)) | |

Results obtained are set forth in Table 6.

TABLE 6
Bioassay Results

| | Day-1 Reading | | | | | Day-7 Reading | | | | |
|---|---|---|---|---|---|---|---|---|---|---|
| | | | | Control | | | | | Control | |
| Treat- | Replicates | | | | STD | Replicate | | | | STD |
| ment | 1 | 2 | 3 | AVG | DEV | 1 | 2 | 3 | AVG | DEV |
| 1 | 0 | 0 | 0 | 0.0 | 0.0 | 0 | 0 | 0 | 0.0 | 0.0 |
| 2 | 25 | 5 | 25 | 18.3 | 11.5 | 25 | 10 | 37.5 | 24.2 | 13.8 |
| 3 | 75 | 75 | 75 | 75.0 | 0.0 | 87.5 | 87.5 | 87.5 | 87.5 | 0.0 |
| 4 | 0 | 0 | 0 | 0.0 | 0.0 | 0 | 0 | 0 | 0.0 | 0.0 |
| 5 | 0 | 0 | 0 | 0.0 | 0.0 | 0 | 0 | 0 | 0.0 | 0.0 |
| 6 | 0 | 0 | 0 | 0.0 | 0.0 | 87.5 | 87.5 | 75 | 83.3 | 7.2 |

Based on the data presented in the table above, hexyl paraben was found to be the most potent herbicidal compound.

Insecticidal Acitivity. The insecticidal activity of butyl paraben (MBI206-FP-F5H32) and hexyl paraben (MBI206-FP-F5H40) were tested in a laboratory assay using a 96-well diet overlay assay with 1st instar Beet Armyworm (Spodoptera exigua) larvae using microtiter plates with 200 ul of solid, artificial Beet Armyworm diet in each well. One hundred (100) microliters of each test sample (containing 40 ug of sample) is pipetted on the top of the diet (one sample in each well), and the sample is let dry under flowing air until the surface is dry. Each sample was tested in six replicates, and water and a commercial Dipel product are used as negative and positive controls, respectively. One first instar larvae of the test insect (Beet armyworm—Spodoptera exiqua) was placed in each well, and the plate was covered with plastic cover with airholes. The plates with insects were incubated at 26° C. for 6 days with daily mortality evaluations. Based on the results presented in Table 7, hexyl paraben and butyl paraben resulted in 71% and 9% mortality, respectively.

TABLE 7
Insecticidal Bioassay data for butyl paraben (MBI206-
FP-F5H32) and hexyl paraben (MBI206-FP-F5H40) against 1^{st}
instar Beet Army Worm (Spodoptera exigua).

| Sample information | Day 3 | Day 4 |
|---|---|---|
| Butyl paraben MBI206-FP-F5H32) @ 40 μg/well | 8.93 | 8.9286 |
| Hexyl parben (MBI206-FP-F5H40) @ 40 μg/well | 50.00 | 70.833 |
| 2% Dipel | 0.00 | 0 |
| 4% Dipel | 25.00 | 25 |
| 8% Dipel | 0.00 | 25 |
| 16% Dipel | 0.00 | 0 |
| 32% Dipel | 0.00 | 0 |
| 64% Dipel | 25.00 | 100 |
| 40% EtOH | 14.29 | 14.286 |
| Dipel | 33.33 | 100 |
| H₂0 | 0.00 | 0 |

Nematicidal Acitivity; In vitro testing of butyl paraben (MBI206-FP-F5H32) and hexyl paraben (MBI206-FP-F5H40): The pure sample of butyl paraben and hexyl paraben was used in an in vitro 96-well plastic cell-culture plate bioassay. 15-20 nematodes in a 50 μl water solution were exposed to 3 μl of a 20 mg/ml peak concentrate for a 24 hour period at 25° C. Once the incubation period was completed, results were recorded based on a visual grading of immobility of the juvenile nematodes (J2's) in each well treated with compounds; each treatment was tested in replicate of 4 wells. Results are shown in Table 8, which shows the results of two different 96-well plate extract bioassays of compounds. Three controls are included in each trial; 1 positive (1% Avid) & 2 negative (DMSO & water). Trials (T1) was carried out using M. incognita nematodes and and trail (T2) was carried out using M. hapla nematodes, the samples were dissolved in 100% DMSO. The hexyl paraben (MBI206-FP-F5H40) showed the excellent control with the immobility of 93.75% against M. incognita as compared to butyl paraben with 81.25% immobility.

TABLE 8
Effect of hexyl paraben and butyl paraben
on M. incognita and M. hapla.

| | % immo- | % immo- | |
|---|---|---|---|
| | bility | bility | Mean % |
| | (trial | (trial | immo- |
| Sample information | # T1) | # T2) | bility |
| MBI206-FP-F5H32 (butyl paraben) | 75 | 87.5 | 81.25 |
| MBI206-FP-F5H40 (hexyl paraben) | 87.5 | 100 | 93.75 |
| Avid (1%) | 75 | 75 | 75 |
| DMSO | 6.25 | 0 | 3.12 |
| Water | 0 | 0 | 0 |

Study of formation of Parabens during formulation of the product. In order to understand the formation of these parabens, the effect of change in alcohol in the formulation was taken into consideration. The different carbon chain alcohols were used in the formulation and the formation of the new parabens were monitored using LCMS.

Four separate formulation experiments were performed using butanol, hexanol, octanol and cetyl alcohol and all other ingradients were kept same. The formulation products were extracted over the period of 2 days and 3 weeks. The crude extract obtained from these formulations were analysed by LCMS. The corresponding parabens formed for all alcohols except for cetyl alcohol. The yield of the parabens was found to be the highest for butyl paraben, followed by hexyl paraben and then octyl paraben for the one day old formulation product. The analysis result even after 3 weeks remain the same order ie, butyl paraben>hexyl paraben>octyl paraben. Thus, the rate of formation of these parabens such as butyl paraben, hexyl paraben & octyl paraben was found to depend on the carbon chain (number of carbon) of the solvent (alcohol) of the corresponding alcohol used in the formulation (butanol (C4)>hexanol (C6)>octanol (C8) etc). The formation of cetyl paraben was not detected till 3 weeks. The yields of these parabens were found to increase over the time.

Another set of experiments were carried out to understand the role of whole cell broth (WCB) in the formation of the new paraben analogues. In 4 different expt. with were carried out with following changes in the formulation—Expt-1: Propyl paraben (No methyl paraben)+WCB+other ingredients; Expt-2: Methyl paraben (No Propyl paraben)+WCB+other ingredients; Expt-3: No parabens (both)+WCB+other ingredients; Expt-4: Methyl Paraben+Propyl paraben+other ingredients+No WCB.

The above formulations were extracted separately and the crude extract obtained were then analysed using LCMS. The formation of the hexyl paraben was observed only in the first two experiments. Thus, these experiments suggested that WCB plays a very important role in the formation of these parabens.

Isolation of Templazole A and B Methods and Materials. The following procedure is used for the purification of Templazole A and B extracted from cell culture of Burkholderia sp (see FIG. 3): The culture broth derived from the 10-L fermentation Burkholderia (A396) in Hy soy growth medium is extracted with Amberlite XAD-7 resin (Asolkar et al., 2006) by shaking the cell suspension with resin at 225 rpm for two hours at room temperature. The resin and cell mass are collected by filtration through cheesecloth and washed with DI water to remove salts. The resin, cell mass, and cheesecloth are then soaked for 2 h in acetone after which the acetone is filtered and dried under vacuum using rotary evaporator to give the crude extract. The crude extract is then fractionated by using reversed-phase C18 vacuum liquid chromatography (H2O/CH3OH; gradient 90:10 to 0:100%) to give 11 fractions. These fractions are then concentrated to dryness using rotary evaporator and the resulting dry residues are screened for biological activity using 96 well plate lettuce seeding assay. The active fractions are then subjected to reversed phase HPLC (Spectra System P4000 (Thermo Scientific) to give pure compounds, which are then screened in above mentioned bioassays to locate/identify the active compounds. To confirm the identity of the compound, additional spectroscopic data such as LC/MS and NMR is recorded.

The active fraction 5 is purified further by using HPLC C-18 column (Phenomenex, Luna 10u C18(2) 100 A, 250×30), water:acetonitrile gradient solvent system (0-10 min; 80% aqueous CH3CN, 10-25 min; 80-65% aqueous CH3CN, 25-50 min; 65-50% aqueous CH3CN, 50-60 min; 50-70% CH3CN, 60-80 min; 70-0% aqueous CH3CN, 80-85 min; 0-20% aqueous CH3CN) at 8 mL/min flow rate and UV detection of 210 nm, to give templazole B, retention time 46.65 min. The other active fraction 7 is also purified using HPLC C-18 column (Phenomenex, Luna 10u C18(2) 100 A, 250×30), water:acetonitrile gradient solvent system (0-10 min; 80% aqueous CH3CN, 10-25 min; 80-60% aqueous CH3CN, 25-50 min; 60-40% aqueous CH3CN, 50-60 min; 40% CH3CN, 60-80 min; 40-0% aqueous CH3CN, 80-85 min; 0-20% aqueous CH3CN) at 8 mL/min flow rate and UV detection of 210 nm, to give templazole A, retention time 70.82 min.

Mass spectroscopy analysis of pure compounds is performed on a Thermo Finnigan LCQ Deca XP Plus electrospray (ESI) instrument using both positive and negative ionization modes in a full scan mode (m/z 100-1500 Da) on a LCQ DECA XPplus Mass Spectrometer (Thermo Electron Corp., San Jose, Calif.). Thermo high performance liquid chromatography (HPLC) instrument equipped with Finnigan Surveyor PDA plus detector, autosampler plus, MS pump and a 4.6 mm×100 mm Luna C18 5 μm column (Phenomenex). The solvent system consists of water (solvent A) and acetonitrile (solvent B). The mobile phase begins at 10% solvent B and is linearly increased to 100% solvent B over 20 min and then kept for 4 min, and finally returned to 10% solvent B over 3 min and kept for 3 min. The flow rate is 0.5 mL/min. The injection volume was 10 μL and the samples are kept at room temperature in an auto sampler. The compounds are analyzed by LC-MS utilizing the LC and reversed phase chromatography. Mass spectroscopy analysis of the present compounds is performed under the following conditions: The flow rate of the nitrogen gas was fixed at 30 and 15 arb for the sheath and aux/sweep gas flow rate, respectively. Electrospray ionization was performed with a spray voltage set at 5000 V and a capillary voltage at 35.0 V. The capillary temperature was set at 400° C. The data was analyzed on Xcalibur software. The active compound templazole A has a molecular mass of 298 and showed m/z ion at 297.34 in negative ionization mode. The LC-MS chromatogram for templazole B suggests a molecular mass of 258 and exhibited m/z ion at 257.74 in negative ionization mode. 1H, 13C and 2D NMR spectra were measured on a Bruker 500 MHz & 600 MHz gradient field spectrometer. The reference is set on the internal standard tetramethylsilane (TMS, 0.00 ppm).

For structure elucidation of templazole A, the purified compound with a molecular weight 298 is further analyzed using a 500 MHz NMR instrument, and has 1H NMR δ values at 8.44, 8.74, 8.19, 7.47, 7.31, 3.98, 2.82, 2.33, 1.08 and has 13C NMR values of δ 163.7, 161.2, 154.8, 136.1, 129.4, 125.4, 123.5, 123.3, 121.8, 121.5, 111.8, 104.7, 52.2, 37.3, 28.1, 22.7, 22.7. Templazole A has UV absorption bands at 226, 275, 327 nm, which suggested the presence of indole and oxazole rings. The molecular formula, C17H18N2O3, was determined by interpretation of 1H, 13C NMR and HRESI MS data m/z 299.1396 (M+H)+(Calcd for C17H19N2O3, 299.1397), which entails a high degree of unsaturation shown by 10 double bond equivalents. The 13C NMR spectrum revealed signals for all 17 carbons, including two methyls, a methoxy, a methylene carbon, an aliphatic methine, an ester carbonyl, and eleven aromatic carbons. The presence of 3′-substituted indole was revealed from 1H-1H COSY and HMBC spectral data. The 1H-1H COSY and HMBC also indicated the presence of a carboxylic acid methyl ester group and a —CH2-CH-(CH3)2 side chain. From the detailed analysis of 1H-1H COSY, 13C, and HMBC data it was derived that the compound contained an oxazole nucleus. From the 2D analysis it was found that the iso-butyl side chain was attached at C-2 position, a carboxylic acid methyl ester at C-4 position and the indole unit at C-5 position to give templazole A.

The second herbicidally active compound, templazole B, with a molecular weight 258 is further analyzed using a 500 MHz NMR instrument, and has 1H NMR δ values at 7.08, 7.06, 6.75, 3.75, 2.56, 2.15, 0.93, 0.93 and 13C NMR values of δ 158.2, 156.3, 155.5, 132.6, 129.5, 129.5, 127.3, 121.8, 115.2, 115.2, 41.2, 35.3, 26.7, 21.5, 21.5. The molecular formula, is assigned as C15H18N2O2, which is determined by interpretation of 1H, 13C NMR and mass data. The 13C NMR spectrum revealed signals for all 15 carbons, including two methyls, two methylene carbons, one aliphatic methine, one amide carbonyl, and nine aromatic carbons. The general nature of the structure was deduced from 1H and 13C NMR spectra that showed a para-substituted aromatic ring [δ 7.08 (2H, d, J=8.8 Hz), 6.75 (2H, d, J=8.8 Hz), and 132.7, 129.5, 115.2, 127.3, 115.2, 129.5]. The 1H NMR spectrum of this structure together with the 1H-1H COSY and HSQC spectra, displayed characteristic signals for an isobutyl moiety [δ 0.93 (6H, d, J=6.9 Hz), 2.15 (1H, sept., J=6.9 Hz), 2.57 (2H, d, J=6.9 Hz). In addition, an olefinic/aromatic proton at (δ 7.06, s), and a carbonyl carbon group (δ 158.9) were also found in the 1H and 13C NMR spectra. On inspection of the HMBC spectrum, the H-1′ signal in the isobutyl moiety correlated with the olefinic carbon (C-2, δ 156.3), and the olefinic proton H-4 correlated with (C-5, δ 155.5; C-2, 156.3 & C-1″, 41.2). The methylene signal at δ 3.75 correlated with C-5, C-4 as well as the C-2″ of the para-substituted aromatic moiety. All these observed correlations suggested the connectivity among the isobutyl, and the para-substituted benzyl moieties for the skeleton of the structure as shown. In addition, the carboxamide group is assigned at the para position of the benzyl moiety based on the HMBC correlation from the aromatic proton at H-4″& H-6″ position. Thus, based on the above data, the structure was designated as templazole B.

Isolation of FR901228. The whole cell broth from the fermentation of Burkholderia sp. in an undefined growth medium is extracted with Amberlite XAD-7 resin (Asolkar et al., 2006) by shaking the cell suspension with resin at 225 rpm for two hours at room temperature. The resin and cell mass are collected by filtration through cheesecloth and washed with DI water to remove salts. The resin, cell mass, and cheesecloth are then soaked for 2 h in acetone after which the acetone is filtered and dried under vacuum using rotary evaporator to give the crude extract. The crude extract is then fractionated by using reversed-phase C18 vacuum liquid chromatography (H2O/CH3OH; gradient 90:10 to 0:100%) to give 11 fractions. These fractions are then concentrated to dryness using rotary evaporator and the resulting dry residues are screened for biological activity using both insect bioassay as well as herbicidal bioassay. The active fractions are then subjected to reversed/normal phase HPLC (Spectra System P4000; Thermo Scientific) to give pure compounds, which are then screened in herbicidal, insecticidal and nematicidal bioassays described below to locate/identify the active compounds. To confirm the identity of the compound, additional spectroscopic data such as LC/MS and NMR is recorded.

Mass spectroscopy analysis of active peaks is performed on a Thermo Finnigan LCQ Deca XP Plus electrospray (ESI) instrument using both positive and negative ionization modes in a full scan mode (m/z 100-1500 Da) on a LCQ DECA XPplus Mass Spectrometer (Thermo Electron Corp., San Jose, Calif.). Thermo high performance liquid chromatography (HPLC) instrument equipped with Finnigan Surveyor PDA plus detector, autosampler plus, MS pump and a 4.6 mm×100 mm Luna C18 5 μm column (Phenomenex). The solvent system consists of water (solvent A) and acetonitrile (solvent B). The mobile phase begins at 10% solvent B and is linearly increased to 100% solvent B over 20 min and then kept for 4 min, and finally returned to 10% solvent B over 3 min and kept for 3 min. The flow rate is 0.5 mL/min. The injection volume is 10 μL and the samples are kept at room temperature in an auto sampler. The compounds are analyzed by LC-MS utilizing the LC and reversed phase chromatography. Mass spectroscopy analysis of the present compounds is performed under the following conditions: The flow rate of the nitrogen gas is fixed at 30 and 15 arb for the sheath and aux/sweep gas flow rate, respectively. Electrospray ionization is performed with a spray voltage set at 5000 V and a capillary voltage at 35.0 V. The capillary temperature is set at 400° C. The data is analyzed on Xcalibur software. Based on the LC-MS analysis, the active insecticidal compound from fraction 6 has a molecular mass of 540 in negative ionization mode.

For structure elucidation, the purified insecticidal compound from fraction 6 with molecular weight 540 is further analyzed using a 500 MHz NMR instrument, and has 1H NMR values at 6.22, 5.81, 5.69, 5.66, 5.65, 4.64, 4.31, 3.93, 3.22, 3.21, 3.15, 3.10, 2.69, 2.62, 2.26, 2.23. 1.74, 1.15, 1.12, 1.05, 1.02; and has 13C NMR values of 172.99, 172.93, 169.57, 169.23, 167.59, 130.74, 130.12, 129.93, 128.32, 73.49, 62.95, 59.42, 57.73, 38.39, 38.00, 35.49, 30.90, 30.36, 29.26, 18.59, 18.38, 18.09, 17.93, 12.51. The NMR data indicates that the compound contains amino, ester, carboxylic acid, aliphatic methyl, ethyl, methylene, oxymethylene, methine, oxymethine and sulfur groups. The detailed 1D and 2D NMR analysis confirms the structure for the compound as FR901228 as a known compound.

Isolation of Templamide A, B, FR901465 and FR901228. The culture broth derived from the 10-L fermentation Burkholderia (A396) in Hy soy growth medium is extracted with Amberlite XAD-7 resin (Asolkar et al., 2006) by shaking the cell suspension with resin at 225 rpm for two hours at room temperature. The resin and cell mass are collected by filtration through cheesecloth and washed with DI water to remove salts. The resin, cell mass, and cheesecloth are then soaked for 2 h in acetone after which the acetone is filtered and dried under vacuum using rotary evaporator to give the crude extract. The crude extract is then fractionated by using reversed-phase C18 vacuum liquid chromatography (H2O/CH3OH; gradient 90:10 to 0:100%) to give 11 fractions. These fractions are then concentrated to dryness using rotary evaporator and the resulting dry residues are screened for biological activity using 96 well plate lettuce seeding (herbicidal) and early 3rd instar Beet Armyworm (insecticidal) assay. The active fractions are then subjected to repeatedly to reversed phase HPLC separation (Spectra System P4000 (Thermo Scientific) to give pure compounds, which are then screened in above-mentioned bioassays to locate/identify the active compounds. To confirm the identity of the compound, additional spectroscopic data such as LC/MS, HRMS and NMR are recorded.

The active fraction 6 is purified further by using HPLC C-18 column (Phenomenex, Luna 10u C18(2) 100 A, 250×30), water:acetonitrile gradient solvent system (0-10 min; 80% aqueous CH3CN, 10-25 min; 80-65% aqueous CH3CN, 25-50 min; 65-50% aqueous CH3CN, 50-60 min; 50-70% aqueous CH3CN, 60-80 min; 70-0% aqueous CH3CN, 80-85 min; 0-20% aqueous CH3CN) at 8 mL/min flow rate and UV detection of 210 nm, to give templamide A, retention time 55.64 min and FR901465, retention time 63.59 min and FR90128, retention time 66.65 min respectively. The other active fraction 6 is also purified using HPLC C-18 column (Phenomenex, Luna 10u C18(2) 100 A, 250×30), water:acetonitrile gradient solvent system (0-10 min; 70-60% aqueous CH3CN, 10-20 min; 60-40% aqueous CH3CN, 20-50 min; 40-15% aqueous CH3CN, 50-75 min; 15-0% CH3CN, 75-85 min; 0-70% aqueous CH3CN) at 8 mL/min flow rate and UV detection of 210 nm, to give templamide B, retention time 38.55 min.

Mass spectroscopy analysis of pure compounds is performed on a Thermo Finnigan LCQ Deca XP Plus electrospray (ESI) instrument using both positive and negative ionization modes in a full scan mode (m/z 100-1500 Da) on a LCQ DECA XPplus Mass Spectrometer (Thermo Electron Corp., San Jose, Calif.). Thermo high performance liquid chromatography (HPLC) instrument equipped with Finnigan Surveyor PDA plus detector, autosampler plus, MS pump and a 4.6 mm×100 mm Luna C18 5 μm column (Phenomenex) is used. The solvent system consists of water (solvent A) and acetonitrile (solvent B). The mobile phase begins at 10% solvent B and is linearly increased to 100% solvent B over 20 min and then kept for 4 min, and finally returns to 10% solvent B over 3 min and kept for 3 min. The flow rate is 0.5 mL/min. The injection volume is 10 μL and the samples are kept at room temperature in an auto sampler. The compounds are analyzed by LC-MS utilizing the LC and reversed phase chromatography. Mass spectroscopy analysis of the present compounds is performed under the following conditions: The flow rate of the nitrogen gas is fixed at 30 and 15 arb for the sheath and aux/sweep gas flow rate, respectively. Electrospray ionization is performed with a spray voltage set at 5000 V and a capillary voltage at 45.0 V. The capillary temperature is set at 300° C. The data is analyzed on Xcalibur software. The active compound templamide A has a molecular mass of 555 based on the m/z peak at 556.41 [M+H]+ and 578.34 [M+Na]+ in positive ionization mode. The LC-MS analysis in positive mode ionization for templamide B suggests a molecular mass of 537 based m/z ions at 538.47 [M+H]+ and 560.65 [M+Na]+. The molecular weight for the compounds FR901465 and FR901228 are assigned as 523 and 540 respectively on the basis of LCMS analysis. 1H, 13C and 2D NMR spectra are measured on a Bruker 600 MHz gradient field spectrometer. The reference is set on the internal standard tetramethylsilane (TMS, 0.00 ppm).

For structure elucidation of templamide A, the purified compound with molecular weight 555 is further analyzed using a 600 MHz NMR instrument, and has 1H NMR δ values at 6.40, 6.39, 6.00, 5.97, 5.67, 5.54, 4.33, 3.77, 3.73, 3.70, 3.59, 3.47, 3.41, 2.44, 2.35, 2.26, 1.97, 1.81, 1.76, 1.42, 1.37, 1.16, 1.12, 1.04 and has 13C NMR values of δ 173.92, 166.06, 145.06, 138.76, 135.71, 129.99, 126.20, 123.35, 99.75, 82.20, 78.22, 76.69, 71.23, 70.79, 70.48, 69.84, 60.98, 48.84, 36.89, 33.09, 30.63, 28.55, 25.88, 20.37, 18.11, 14.90, 12.81, 9.41. The 13C NMR spectrum exhibits 28 discrete carbon signals which are attributed to six methyls, four methylene carbons, and thirteen methines including five sp2, four quaternary carbons. The molecular formula, C₂₈H₄₅NO₁₀, is determined by interpretation of 1H, 13C NMR and HRESI MS data. The detailed analysis of 1H-1H COSY, HMBC and HMQC spectral data reveals the following substructures (I-IV) and two isolated methylene & singlet methyl groups. These substructures are connected later using the key HMBC correlations to give the planer structure for the compound, which has been not yet reported in the literature and designated as templamide A. This polyketide molecule contains two tetrahydropyranose rings, and one conjugated amide.

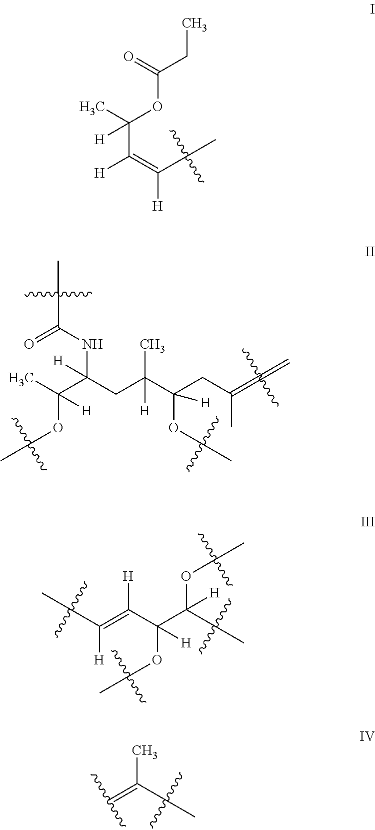

Substructures I-IV assigned by analysis of 1D & 2D NMR spectroscopic data.

The (+) ESIMS analysis for the second herbicidal compound, shows m/z ions at 538.47 [M+H]+ and 560.65 [M+Na]+ corresponding to the molecular weight of 537. The molecular formula of C28H43NO9 is determined by interpretation of the ESIMS and NMR data analysis. The 1H and 13C NMR of this compound is similar to that of templamide A except that a new isolated —CH2- appear instead of the non-coupled methylene group in templamide A. The small germinal coupling constant of 4.3 Hz is characteristic of the presence of an epoxide methylene group. The presence of this epoxide is further confirmed from the 13C NMR shift from 60.98 in templamide A to 41.07 in compound with MW 537. The molecular formulae difference between these two compounds is reasonably explained by elimination of the water molecule followed by formation of epoxide. Thus, on the basis of based NMR and MS analysis the structure for the new compound was assigned and was designated as templamide B.

For structure elucidation, the purified compound from fraction 6 with molecular weight 523 is further analyzed using a 600 MHz NMR instrument, and has 1H NMR values at 6.41, 6.40, 6.01, 5.98, 5.68, 5.56, 4.33, 3.77, 3.75, 3.72, 3.65, 3.59, 3.55, 3.50, 2.44, 2.26, 2.04, 1.96, 1.81, 1.75, 1.37, 1.17, 1.04; and has 13C NMR values of 172.22, 167.55, 144.98, 138.94, 135.84, 130.14, 125.85, 123.37, 99.54, 82.19, 78.28, 76.69, 71.31, 70.13, 69.68, 48.83, 42.52, 36.89, 33.11, 30.63, 25.99, 21.20, 20.38, 18.14, 14.93, 12.84. The detailed 1H and 13C NMR analysis of compound suggested that this compound was quite similar to compound templamide B; the only difference was in the ester side chain; an acetate moiety was present instead of a propionate moiety in the side chain. The detailed 1D and 2D NMR analysis confirm the structure for the compound as FR901465 as a known compound.

Based on the LC-MS analysis, the other compound from fraction 6 has a molecular mass of 540 in negative ionization mode. For structure elucidation, the purified compound from fraction 5 with molecular weight 540 is further analyzed using a 500 MHz NMR instrument, and has 1H NMR values at 6.22, 5.81, 5.69, 5.66, 5.65, 4.64, 4.31, 3.93, 3.22, 3.21, 3.15, 3.10, 2.69, 2.62, 2.26, 2.23. 1.74, 1.15, 1.12, 1.05, 1.02; and has 13C NMR values of 172.99, 172.93, 169.57, 169.23, 167.59, 130.74, 130.12, 129.93, 128.32, 73.49, 62.95, 59.42, 57.73, 38.39, 38.00, 35.49, 30.90, 30.36, 29.26, 18.59, 18.38, 18.09, 17.93, 12.51. The NMR data indicates that the compound contains amino, ester, carboxylic acid, aliphatic methyl, ethyl, methylene, oxymethylene, methine, oxymethine and sulfur groups. The detailed 1D and 2D NMR analysis confirm the structure for the compound as FR901228 as a known compound.

The molecular weight for the other active compound (F8H17) from Fraction F8 was assigned as 1080 based on the molecular ion peak at 1081.75 (M+H) in positive ESI mode and further confirmed by the negative ESIMS with base peak at 1079.92. This compound showed UV absorption at 234 nm.

Burkholderia sp. as an Algicide. Burkholderia sp. A396 is grown in an undefined mineral medium for 5 days (25° C., 200 rpm). Cells are separated from the supernatant by centrifugation at 8,000 g, and the cell-free supernatant is used to test the algaicidal activity against a unicellular algal species (P. subcapitata) and a blue-green alga species (Anabaena sp.). A specified increasing amount of supernatant is added into wells of a 24-well polystyrene plate that has the specified algae growing in 750 micro liters of Gorham's medium to determine the dose-response curve for the test supernatant on each algae type. Each treatment is done in two replicates, and the blank growth medium is used as a negative control. The plate is closed with a lid and incubated for 48 hours under constant growth light at room temperature. After 48 hours, the fluorescence (at 700 nm) of the suspension in each well is measured using a SpectraMax Gemini XS plate reader, and the reduction in fluorescence compared with the un-treated control is converted into percent control of algal growth. Results presented in Table 9 below show excellent control of unicellular algae and good control or algistatic effect on blue-green algae.

TABLE 9
Control of two algal species by Burkholderia A396 cell-free
broth measured as a reduction of fluorescence at 700 nm.

% control

| Amount of broth per well | P. subcapitata | Anabaena sp. |
|---|---|---|
| 0 μL | 0 | 0 |
| 10 μL | 74.2 | 0.0 |
| 20 μL | 84.1 | 0.0 |
| 30 μL | 85.5 | 0.0 |
| 40 μL | 88.3 | 0.0 |
| 50 μL | 90.6 | 0.0 |
| 100 μL | 94.6 | 36.4 |

Control of Chlamydomonas reinhardtii by crude extract and fractions of Burkholderia sp. Fractions obtained from the fractionation of crude extract of Burkholderia sp. were tested for algaecide activity against Chlamydomonas reinhardtii. An increasing volume of fraction (with concentration of 20 mg/mL in ethanol) was added to a clear 48 well polystyrene plate with 750 micro liters of the specified algae growing. Each treatment was done in two replicates and the solvent (ethanol) used as a negative control. The plate was closed with a lid and incubated for 72 hours under constant light at room temperature. After 72 hours, the fluorescence (at 680 nm) of the suspension in each well was measured using a SpectraMax M2 plate reader, and the reduction in fluorescence compared with the negative control was converted into percent control of algal growth. Each sample was visually compared to the negative control; a well that was visually clearer than the negative control was scored as active. Results presented in Table 10 below shows control of the specified algae in fractions 5, 6, 7, 8, and 9. Tests were run in two replicates and % Control was calculated as a reduction of fluorescence at 680 nm compared with the negative control. Each sample was visually compared to the negative control; a well that was visually clearer than the negative control was scored as active.

TABLE 10
Control of Chlamydomonas reinhardtii by crude extract
& fractions of Burkholderia sp. (MBI 206).

| | μL of Sample per 750 | | |
|---|---|---|---|
| Sample | μL of Algae | % Inhibition | Visual |
| Solvent Blank | 22.5 | 0.00 | Not Active |
| | 11 | 0.00 | Not Active |
| | 5 | 0.00 | Not Active |
| Crude Extract | 22.5 | 97.10 | Active |
| | 11 | 89.54 | Active |
| | 5 | 90.82 | Active |
| MBI 206F1 | 22.5 | −74.47 | Not Active |
| | 11 | 46.47 | Not Active |
| | 5 | 46.21 | Not Active |
| MBI 206F2 | 22.5 | 12.64 | Not Active |
| | 11 | −214.35 | Not Active |
| | 5 | −297.56 | Not Active |
| MBI 206F3 | 22.5 | −143.92 | Not Active |
| | 11 | −740.16 | Not Active |
| | 5 | 32.68 | Not Active |
| MBI 206F4 | 22.5 | −98.80 | Not Active |
| | 11 | −155.41 | Not Active |
| | 5 | 58.51 | Not Active |
| MBI 206F5 | 22.5 | 92.89 | Active |
| | 11 | 79.45 | Active |
| | 5 | 71.60 | Weak |
| MBI 206F6 | 22.5 | 94.88 | Active |
| | 11 | 96.33 | Active |
| | 5 | 86.45 | Active |
| MBI 206F7 | 22.5 | 97.32 | Active |
| | 11 | 98.96 | Active |
| | 5 | 97.89 | Active |
| MBI 206F8 | 22.5 | 94.35 | Active |
| | 11 | 32.17 | Weak |
| | 5 | −13.51 | Not Active |
| MBI 206F9 | 22.5 | 85.35 | Active |
| | 11 | 96.49 | Active |
| | 5 | 97.73 | Active |
| MBI 206F10 | 22.5 | 50.30 | Not Active |
| | 11 | 48.54 | Not Active |
| | 5 | −108.24 | Not Active |
| MBI 206F11 | 22.5 | −121.50 | Not Active |
| | 11 | −16.21 | Not Active |
| | 5 | 36.46 | Not Active |

Algicidal effect of crude extract and various fractions obtained from Burkholderia sp. against P. subcapitata. The crude extract as well as the fractions obtained from Burkholderia sp. was tested for algicidal activity against a unicellular algal species (P. subcapitata). An increasing volume of pure ethanol solution derived by re-dissolving a known amount of material (10 mg/mL concentration) corresponding to each sample was added into wells of a 24-well polystyrene plate that has the specified algae growing in 750 micro liters of Gorham's medium to determine the algicidal effect of sample (extract/fractions) on unicellular algae. Each treatment was done in three replicates, and pure ethanol was used as a negative control. After mixing, the plate was closed with a lid and incubated for 48 hours under constant growth lights at room temperature. After 48 hours, the fluorescence (at 700 nm) of the suspension in each well was measured using a SpectraMax Gemini XS plate reader, and the reduction in fluorescence compared with the un-treated control was converted into percent control of algal growth. Results presented in Table 11 below show excellent control of unicellular algae with fractions F5, F6 and F7 whereas no substantial algicidal effect was obtained with other samples.

TABLE 11
Algicidal effect of various samples obtained from
Burkholderia sp. The bioassay was run in three replicates
using P. subcapitata as the test organism.

| Sample | sample μL | % control | Results |
|---|---|---|---|
| MBI206F1 | 5 | 0.0 | Not Active |
| | 10 | 33.9 | Not Active |
| | 20 | 58.2 | Not Active |
| MBI206F2 | 5 | 35.7 | Not Active |
| | 10 | 6.0 | Not Active |
| | 20 | 31.8 | Not Active |
| MBI206F3 | 5 | 40.9 | Not Active |
| | 10 | 66.4 | Not Active |
| | 20 | 68.5 | Not Active |
| MBI206F4 | 5 | 46.8 | Not Active |
| | 10 | 69.8 | Weak |
| | 20 | 84.7 | Active |
| MBI206F5 | 5 | 49.9 | Not Active |
| | 10 | 71.5 | weak |
| | 20 | 95.4 | Active |
| MBI206F6 | 5 | 62.7 | Not Active |
| | 10 | 74.7 | weak |
| | 20 | 90.7 | Active |
| MBI206F7 | 5 | 40.1 | Not Active |
| | 10 | 88.6 | Active |
| | 20 | 93.0 | Active |
| MBI206F8 | 5 | 36.8 | Not Active |
| | 10 | 50.0 | Not Active |
| | 20 | 65.9 | Not Active |
| MBI206F9 | 5 | 66.3 | Not Active |
| | 10 | 40.7 | Not Active |
| | 20 | 51.8 | Not Active |
| MBI206F10 | 5 | 26.8 | Not Active |
| | 10 | 27.5 | Not Active |
| | 20 | 32.9 | Not Active |
| MBI206F11 | 5 | 25.9 | Not Active |
| | 10 | 32.8 | Not Active |
| | 20 | 39.2 | Not Active |
| Crude extract | 5 | 45.6 | Not Active |
| | 10 | 69.6 | weak |
| | 20 | 70.0 | weak |
| Solvent Blank | 5 | 0.0 | Not Active |
| | 10 | 0.0 | Not Active |
| | 20 | 0.0 | Not Active |

Control of Chlamydomonas reinhardtii by purified compounds from Burkholderia sp. fermentation broth Purified compounds from Burkholderia sp. fermentation broth was tested for algaicidal activity against Chlamydomonas reinhardtii. An increasing volume of the purified compounds (20 mg/mL in ethanol) was added to a clear 48 well polystyrene plate with 750 micro liters of the specified algae growing. Each treatment was done in two replicates and the solvent used as a negative control. The plate was closed with a lid and incubated for 72 hours under constant light at room temperature. After 72 hours the fluorescence (at 680 nm) of the suspension in each well was measured using a SpectraMax M2 plate reader, and the reduction in fluorescence compared with the negative control was converted into percent control of algal growth. Each sample was visually compared to the negative control; a well that was visually clearer than the negative control was scored as active. Results presented in Table 12 below shows control of the specified algae in samples containing templamide B (MW 537), FR901228 (MW 540), templazole A (MW 298), and F8H18 (MW 1080). Tests were run in two replicates and %. Control was calculated as a reduction of fluorescence at 680 nm compared with the negative control. Each sample was visually compared to the negative control; a well that was visually clearer than the negative control was scored as active.

TABLE 12
Control of Chlamydomonas reinhardtii by purified compounds
from Burkholderia sp. fermentation broth (MBI 206).

| | μL of Sample | | |
|---|---|---|---|
| | per 750 μL of | | |
| Sample | Algae | % Control | Visual |
| Solvent Blank | 22.5 | 0 | Not Active |
| | 11 | 0 | Not Active |
| | 5 | 0 | Not Active |
| Templamide B | 22.5 | 98.29620264 | Active |
| (MW 537) | 11 | 99.34438783 | Active |
| | 5 | 95.05204335 | Active |
| FR901465 | 22.5 | −3.887169203 | Not Active |
| (MW 523) | 11 | −33.58351827 | Not Active |
| | 5 | −86.58233289 | Not Active |
| Templamide A | 22.5 | −151.6466844 | Not Active |
| (MW 555) | 11 | −21.16166036 | Not Active |
| | 5 | −67.61183948 | Not Active |
| FR901228 | 22.5 | 98.71299647 | Active |
| (MW 540) | 11 | 99.35532773 | Active |
| | 5 | 89.48079462 | Active |
| Templazole B | 22.5 | −30.78693813 | Not Active |
| (MW 258) | 11 | 52.94712906 | Not Active |
| | 5 | −102.0883867 | Not Active |
| Templazole A | 22.5 | 98.1523303 | Active |
| (MW 298) | 11 | 98.72823743 | Active |
| | 5 | 99.18429591 | Active |
| Templazole A | 22.5 | 95.71173214 | Active |
| (MW 298) | 11 | 98.31330291 | Active |
| | 5 | 98.69251947 | Active |
| F8H18 | 22.5 | 94.98474386 | Active |
| (MW 1080) | 11 | 82.90378804 | Active |
| | 5 | −21.38764258 | Not Active |

Templazole A was tested twice in this bioassay.

Control of Scenedesmus quadricauda by heat-treated Burkholderia sp. fermentation supernatant. Burkholderia sp. was grown in a fermentation broth as previously described. The broth was heat treated at the end of the fermentation to inactivate all cells. The cell free supernatant was tested for algaecide activity against Scenedesmus quadricauda. An increasing volume of supernatant was added to a clear 48 well polystyrene plate with 750 micro liters of the specified algae growing. Each treatment is done in two replicates and the blank growth medium used as a negative control. The plate is closed with a lid and incubated for 72 hours under constant light at room temperature. After 72 hours the fluorescence (at 680 nm) of the suspension in each well is measured using a SpectraMax M2 plate reader, and the reduction in fluorescence compared with the untreated control is converted into percent control of algal growth. Results presented in Table 13 below shows control of the specified algae. Tests were run in two replicates and % Control was calculated as a reduction of fluorescence at 680 nm compared with the untreated control.

TABLE 13
Control of Scenedesmus quadricauda by supernatant
of heat kill Burkholderia sp. (MBI 206).

| Material | Volume (μL) | % Inhibition |
|---|---|---|
| MBI 206 | 0 | 0 |
| 120522ST HK | 10 | 97.21347952 |
| TGAI | 20 | 99.36167161 |
| | 30 | 99.42844203 |
| | 40 | 99.50798231 |
| | 50 | 98.90136045 |
| | 100 | 95.9474484 |

Control of Oscillatoria tenius by heat kill Burkholderia sp. fermentation supernatant. Burkholderia sp. was grown in a fermentation broth as previously described. The broth was heat treated at the end of the fermentation to inactivate all cells. The cell free supernatant was tested for algaecide activity against Oscillatoria tenius. An increasing volume of supernatant was added to a clear 48 well polystyrene plate with 750 μL of the specified algae growing. Each treatment is done in two replicates and the blank growth medium used as a negative control. The plate is closed with a lid and incubated for 72 hours under constant light at room temperature. After 72 hours the absorbance at 680 nm is measured in each well using a SpectraMax M2 plate reader, and the reduction in absorbance compared with the untreated control is converted into percent control of algal growth. Results presented in Table 14 below shows control of the specified algae. Tests were run in two replicates and % control was calculated as a reduction of absorbance at 680 nm compared with the untreated control.

TABLE 14
Control of Oscillatoria tenius by supernatant
of heat kill Burkholderia sp. (MBI 206).

| Material | Volume (μL) | % Control |
|---|---|---|
| MBI 206 | 0 | 0 |
| 120522ST HK | 10 | 6.177042802 |
| TGAI | 20 | 25.12413108 |
| | 30 | 10.56583534 |
| | 40 | 37.70086527 |
| | 50 | 45.47313627 |
| | 100 | 36.96205601 |

Efficacy of Burkholderia sp. against two-spotted spidermites infesting marigold plants. Marigold, Tagetes erecta, grown in 6″ containers were infested with two-spotted spidermite, Tetranychus urticae, by placing leaves extracted from host plant (cotton) onto the test plants. Approximately ten (10) leaves with 30-40 spidermites present were placed on various parts of test plants for fourteen (14) days. Test plants were individually caged following infestation to allow spidermite population to build. Host leaves were removed from test plant. No pesticides were applied to test plants prior to study application. Spray application was applied using a Gen3 spray booth calibrated to 100 gpa. Each replicate was individually caged immediately following application. Cage description; a wire tomato cage 30″ height×12″ diameter, covered with antivirus insect screening Test plants received natural lighting for duration of trial. Test plants were soil watered every twenty-four (24) hours as needed. Plants were evaluated prior to application (pre-count), 3, 5 and 7 days after application. Four leaves were randomly selected and harvested from each replicate equaling a 6 cm sq total surface area evaluated. Actual count was recorded on live and dead two-spotted spidermite. Burkholderia sp. showed slight activity against both TSSM nymphs and adults. This activity shows potential for biopesticide formulations against TSSM. The treatments also reduced the number of live mites observed on samples. This is compelling evidence that MBI206 shows potential for biopesticide formulations against TSSM.

Efficacy of Burkholderia sp. fermentation supernatant against two-spotted spidermites infesting Marigold plants. Marigold plants grown in 6″ containers were infested with two-spotted spidermite by placing leaves extracted from host plant (cotton) onto the test plants. Eight to ten (8-10) leaves with approximately 30-40 two-spotted spidermite present were placed on various parts of test plants for fourteen (14) days. Test plants were individually caged following infestation to allow mite population to build. Host leaves were removed from test plant. No pesticides were applied to test plants prior to study application. Plants were treated with either 100% supernatant or 10% supernatant (in water) Spray application was applied to full coverage with no run-off using a disposable hand-sprayer. Test plants were placed research greenhouse on a wire-mesh raised bench and arranged in a complete randomized block design. Research greenhouse is monitored by Procom, Micro Grow Greenhouse System temperature control system. Environmental conditions averaged high temperature 85 F to low temperature of 72 F during trial dates. Average humidity levels ranged from 40% to 75%. Test plants received natural lighting for duration of trial. Test plants were soil watered every twenty-four (24) hours as needed. Plants were evaluated prior to application (pre-count), 3, 5, 7 and 14 days after application. Evaluations were taken on a 6 cm square total area per replicate. Actual count was recorded on live/dead two-spotted spidermite nymph and live/dead two-spotted spidermite adult.

Example 14: Efficacy of Burkholderia sp. formulation (MBI 206) for control of two spotted spidermite (TSM) in strawberry—field data. The efficacies of five traditional chemistry-derived and MBI 206 were evaluated for TSM control under field conditions. ‘Strawberry Festival’ transplants were set in the field in plastic mulched beds, 13 inches high and 27 inches across the top, and with 4 ft bed spacing. Overhead irrigation was applied for 10 days after setting to aid in establishment of the transplants. Trickle irrigation was used for the remainder of the experiment. Each 12.5-ft. plot consisted of 20 plants in two ten-plant rows per bed. Plots were infested from a laboratory colony in four sessions with 10 to 20 motile TSM, per plant. Each session accomplished the infestation of one block of the experiment. The experiment consisted of treatments of various rates and schedules of application of miticides, some combined with an adjuvant, and a non-treated check. Treatments were replicated four times in a RCB design. Savey and Acramite treatments were applied before TSM densities reached threshold levels (6 Jan); the remainder of the treatment programs began 2 wks later. Treatments were applied using a hand-held sprayer with a spray wand outfitted with a nozzle containing a 45-degree core and a number four disc. The sprayer was pressurized by CO2, to 40 psi, and calibrated to deliver 100 gal per acre. Pre-treatment samples were taken on Day 1 and sampling continued weekly through 2 wks after the last application of treatments. Samples consisted of ten randomly selected leaflets per plot and were collected from the middle one-third stratum of the plants. Samples were transported to the laboratory where motile and egg TSM were brushed from the leaflets onto rotating sticky discs and counted on 1/10 of the disc surface to estimate average numbers per leaflet. Distinctions could not be made between viable and non-viable eggs, thus total eggs were recorded. MBI 206 at the highest rate (3 gal/acre) shows decrease in the number of eggs at a level comparable to at least two of the chemical controls.

Example 15: Control of citrus rust mites (Phyllocoptruta oleivora) on citrus under filed conditions. MBI 206 (formulated broth of Burkhoderia sp.) was sprayed on Valencia Sweet Orange at 1, 2, and 3 gal/acre in combination with 0.25% v/v/ of LI-700 (surfactant) and delivered in a volume of 100 GPA. A single treatment was delivered and compared to an untreated sample. Mite counts were performed pre-treatment, and then at 1, 7, 10 and 14 days after treatment. Mite counts were an average of 10 fruits per treatment per sampling point. A reduction in the number of mites present in the MBI 206 treatments was observed at 14 days after treatments with 1 and 2 gal/acre MBI 206 (approximately 6-8 mites per count), when compared to the untreated control (approx. 16 mites per count).

Example 16: Insecticidal (sucking contact) activity of Templamide, FR901465 and FR901228 against milkweed bugs. The insecticidal activity of the pure compounds templamide B (MBI 206; MW 537), FR 901465 (MBI 206; MW 523) and FR901228 (MBI 206; MW 540) were tested in a laboratory assay using a sucking contact bioassay system. The compounds were dissolved in 100% ethanol to concentrations of 1 mg/mL. Individual 4th instar milkweed bugs, penultimate nymph, larvae were placed in 5C Rubbermaid container with 2 sunflower seeds in each tub and 1 water cup (water in contact cup with cotton wick) into each tub. A Hamilton Micropipette was used to apply 1 μL (1 drop) of compound onto abdomen of milkweed bugs (MWB) of each larvae. Tubs were place into the Rubbermaid container and cap with mesh lid. Eight larvae per sample were treated. The assay was incubated at 25° C., 12 h light/12 h dark. Larvae were scored at 4 and 7 day after application. All the three compounds exhibited contact activity against MWB, while not all insects died but many were clearly affected and unable to move. Most of the MWB on day 7 had molted which suggests that the compounds may inhibit molting or affect normal MWB development. Thus, FR 901465 provided a better (87.5%) control of milkweed bugs, than FR 901228 (MW 540) and templamide B (FIG. 4).

Insecticidal activity of pure compounds against Lygus hesperus late 2nd/early 3rd instar. The insecticidal activity of the four compounds, templamide A, templamide B, FR901465 & FR901228 isolated from Burkholderia were tested in a laboratory assay using a 12 well plate with treated green beans bioassay system. The compound was dissolved in 100% ethanol to concentrations of 1 mg/mL and 500 μL of this sample was added to 3.5 mL of water to make a total volume of 4 mL containing 0.25 mg/mL concentration of the compound. Green beans were washed earlier in bleach solution and then sat in water to rinse. Beans were dried before using and then were cut with scissors to fit into wells of 12-well plate. With the help of forceps the beans were dunked into a 15 mL plastic falcon tube containing each treatment and then submerged in treatment for exactly one min. one bean part was put into each well and then individual late 2nd/early 3rd instar Lygus hesperus, were placed in wells with help of brush. Plate sealer was used to cover tray and hole poked into the plate sealer for aeration. The numbers of Lygus/well were counted and plates were placed on brench top. Larvae were scored at 24, 48 and 120 hours after application. Based on the results presented in FIG. 5, compound FR 901465, was found to be the most potent with mortality of 91.2%, followed by templamide with B 69.2%, and FR901228 with 51.7%. The templamide A was inactive in the Lygus feeding bioassay. The positive control used in this testing was Avid (Avemectin) at the rate of 13 μL/10 mL.

Nematicidal Activity of FR901228. The pure sample of FR 901228 was tested using an in vitro 96-well plastic cell-culture plate bioassay. 15-20 nematodes in a 50 μl water solution were exposed to 3 μl of a 20 mg/ml solution of FR 901228 for a 24 hour period at 25 C. Once the incubation period was completed, results were recorded based on a visual grading of immobility of the juvenile nematodes (J2's) in each well treated with compounds; each treatment was tested in replicate of 4 wells. Three controls are included in each trial; 1 positive (1% Avid) & 2 negative (DMSO & water). Trials (T1) was carried out using Free living nematodes (FLN) and trail (T2) was carried out using M. incognita nematodes, the samples were dissolved in 100% DMSO. FR 901228 (MW 540) showed the excellent control with immobility of 75% against free living nematodes as compared to M. incognita with 75% immobility.

The following biological material has been deposited under the terms of the Budapest Treaty with the Agricultural Research Culture Collection (NRRL), 1815 N. University Street, Peoria, Ill. 61604 USA, and given the following number:

| Deposit | Accession Number | Date of Deposit |
|---|---|---|
| Burkholderia sp. A396 | NRRL B-50319 | Sep. 15, 2009 |

The strain has been deposited under conditions that assure that access to the culture will be available during the pendency of this patent application to one determined by the Commissioner of Patents and Trademarks to be entitled thereto under 37 C.F.R. §1.14 and 35 U.S.C. §122. The deposit represents a substantially pure culture of the deposited strain. The deposit is available as required by foreign patent laws in countries wherein counterparts of the subject application, or its progeny are filed. However, it should be understood that the availability of a deposit does not constitute a license to practice the subject invention in derogation of patent rights granted by government action.

Although this invention has been described with reference to specific embodiments, the details thereof are not to be construed as limiting, as it is obvious that one can use various equivalents, changes and modifications and still be within the scope of the present invention. Various references are cited throughout this specification, each of which is incorporated herein by reference in its entirety.

What is claimed is:

1. A method of modulating the proliferation of pest infestation comprising the steps of:
providing an insecticidal or herbicidal composition comprising: an isolated strain of Burkholderia sp. A 396 (NRRL Accession No. B-50319); n-hexanol; and hexyl paraben formed by incubating isolated strain of Burkholderia sp. A 396 (NRRL Accession No. B-50319) in n-hexanol; and
applying the insecticidal or herbicidal composition to a location where modulation the proliferation of pests is desired an amount of affective to modulate the proliferation of the pest.

2. The method to claim 1, wherein said hexyl paraben is present in the amount of about 0.01-5%, and the n-hexanol is present in the amount of about 0.001-10%.

3. The method of claim 1, wherein said pest is a nematode.

4. The method of claim 1, wherein said pest is a monocot, a dicot, or sedge weeds.

5. The method of claim 1, wherein said pest is a monocot.

6. The method of claim 1, wherein said pest is a dicot.

7. The method of claim 1, wherein said pest is sedge weeds.

8. The method of claim 1, wherein the modulation results in a decrease in the proliferation of the pest.

9. The method of claim 1, wherein the isolated strain of Burkholderia sp. A 396 (NRRL Accession No. B-50319) is lysed.

10. A method for obtaining a hexyl paraben composition comprising
providing a composition comprising an isolated strain of Burkholderia sp. A 396 (NRRL Accession No. B-50319);
incubating the composition in n-hexanol for a time and at a temperature sufficient to produce said hexyl paraben composition.

11. The method of claim 10, wherein said hexyl paraben is present in the amount of about 0.01-5%.

12. The method of claim 10, wherein said n-hexanol is present in the amount of about 0.001-10%.

13. The method of claim 10, further comprising the step of adding a carrier to the hexyl paraben composition to form a compostion for modulating the proliferation of a pest infestation.

14. The method to claim 13, wherein said hexyl paraben is present in the amount of about 0.01-5%.

15. The method of claim 10, wherein said composition is a cell-free supernatant.

16. The method of claim 10, wherein said composition comprises lysed Burkholderia sp. A 396 (NRRL Accession No. B-50319).


SEQUENCE LISTING (15 sequences)

SEQ ID NO: 1 - DNA, 20 bp, Artificial Sequence
  Primer
agagtttgatcctggctcag

SEQ ID NO: 2 - DNA, 20 bp, Artificial Sequence
  Primer
ccgtcaattcctttgagttt

SEQ ID NO: 3 - DNA, 16 bp, Artificial Sequence
  Primer
gtgccagccgccgcgg

SEQ ID NO: 4 - DNA, 16 bp, Artificial Sequence
  Primer
gcaacgagcgcaaccc

SEQ ID NO: 5 - DNA, 17 bp, Artificial Sequence
  Primer
aaggaggtgwtccarcc

SEQ ID NO: 6 - DNA, 15 bp, Artificial Sequence
  Primer
gggttgcgctcgttg

SEQ ID NO: 7 - DNA, 18 bp, Artificial Sequence
  Primer
gwattaccgcggckgctg

SEQ ID NO: 8 - DNA, 871 bp, Burkholderia
  strain A396
tgcagtcgaacggcagcacgggtgcttgcacctggtggcgagtggcgaacgggtgagtaatacatcggaacatgtcctgtagtgggggatagcccggcgaaagccggattaataccgcatacgatctacggatgaaagcgggggatcttcggacctcgcgctatagggttggccgatggctgattagctagttggtggggtaaaggcctaccaaggcgacgatcagtagctggtctgagaggacgatcagccacactgggactgagacacggcccagactcctacgggaggcagcagtggggaattttggacaatgggggaaaccctgatccagcaatgccgcgtgtgtgaagaaggccttcgggttgtaaagcacttttgtccggaaagaaatcctttgggctaataccccggggggatgacggtaccggaagaataagcaccggctaactacgtgccagcagccgcggtaatacgtagggtgcgagcgttaatcggaattactgggcgtaaagcgtgcgcaggcggtttgttaagacagatgtgaaatccccgggcttaacctgggaactgcatttgtgactggcaagctagagtatggcagaggggggtagaattccacgtgtagcagtgaaatgcgtagagatgtggaggaataccgatggcgaaggcagccccctgggccaatactgacgctcatgcacgaaagcgtggggagcaaacaggattagataccctggtagtccacgccctaaacgatgtcaactagttgttggggattcatttccttagtaacgtagctacgcgtgaagttgaccgcctggggagtacggtcgcaagattaaatmgagggtkgkktgkkggggggaaa

SEQ ID NO: 9 - DNA, 1453 bp, Burkholderia
  strain A396
gtcatgaatcctaccgtggtgaccgtcctccttgcggttagactagccacttctggtaaaacccactcccatggtgtgacgggcggtgtgtacaagacccgggaacgtattcaccgcggcatgctgatccgcgattactagcgattccagcttcatgcactcgagttgcagagtgcaatccggactacgatcggttttctgggattagctccccctcgcgggttggcaaccctctgttccgaccattgtatgacgtgtgaagccctacccataagggccatgaggacttgacgtcatccccaccttcctccggtttgtcaccggcagtctccttagagtgctcttgcgtagcaactaaggacaagggttgcgctcgttgcgggacttaacccaacatctcacgacacgagctgacgacagccatgcagcacctgtgtatcggttctctttcgagcactcccgaatctcttcaggattccgaccatgtcaagggtaggtaaggtttttcgcgttgcatcgaattaatccacatcatccaccgcttgtgcgggtccccgtcaattcctttgagttttaatcttgcgaccgtactccccaggcggtcaacttcacgcgttagctacgttactaaggaaatgaatccccaacaactagttgacatcgtttagggcgtggactaccagggtatctaatcctgtttgctccccacgctttcgtgcatgagcgtcagtattggcccagggggctgccttcgccatcggtattcctccacatctctacgcatttcactgctacacgtggaattctacccccctctgccatactctagcttgccagtcacaaatgcagttcccaggttaagcccggggatttcacatctgtcttaacaaaccgcctgcgcacgctttacgcccagtaattccgattaacgctcgcaccctacgtattaccgcggctgctggcacgtagttagccggtgcttattcttccggtaccgtcatccccccggggtattagcccaaaggatttctttccggacaaaagtgctttacaacccgaaggccttcttcacacacgcggcattgctggatcagggtttcccccattgtccaaaattccccactgctgcctcccgtaggagtctgggccgtgtctcagtcccagtgtggctgatcgtcctctcagaccagctactgatcgtcgccttggtaggcctttaccccaccaactagctaatcagccatcggccaaccctatagcgcgaggtccgaagatcccccgctttcatccgtagatcgtatgcggtattaatccggctttcgccgggctatcccccactacaggacatgttccgatgtattactcacccgttcgccactcgccaccaggtgcaagcacccgtgctgccgttcgacttgcatgtgtaaggcatgccgccagcgttcaatctgag

SEQ ID NO: 10 - DNA, 860 bp, Burkholderia
  strain A396
ccaggcggtcacttcacgcgttagctacgttactaaggaaatgaatccccaacaactagttgacatcgtttagggcgtggactaccagggtatctaatcctgtttgctccccacgctttcgtgcatgagcgtcagtattggcccagggggctgccttcgccatcggtattcctccacatctctacgcatttcactgctacacgtggaattctacccccctctgccatactctagcttgccagtcacaaatgcagttcccaggttaagcccggggatttcacatctgtcttaacaaaccgcctgcgcacgctttacgcccagtaattccgattaacgctcgcaccctacgtattaccgcggctgctggcacgtagttagccggtgcttattcttccggtaccgtcatccccccggggtattagcccaaaggatttctttccggacaaaagtgctttacaacccgaaggccttcttcacacacgcggcattgctggatcagggtttcccccattgtccaaaattccccactgctgcctcccgtaggagtctgggccgtgtctcagtcccagtgtggctgatcgtcctctcagaccagctactgatcgtcgccttggtaggcctttaccccaccaactagctaatcagccatcggccaaccctatagcgcgaggtccgaagatcccccgctttcatccgtagatcgtatgcggtattaatccggctttcgccgggctatcccccactacaggacatgttccgatgtattactcacccgttcgccactcgccaccaggtgcaagcacccgtgctgccgttcgacttgcatgtgtaaggcatgccgccagcgttcaatctgagtg

SEQ ID NO: 11 - DNA, 1152 bp, Burkholderia
  strain A396
tcggattactgggcgtaagcgtgcgcaggcggtttgttaagacagatgtgaaatccccgggcttaacctgggaactgcatttgtgactggcaagctagagtatggcagaggggggtagaattccacgtgtagcagtgaaatgcgtagagatgtggaggaataccgatggcgaagggagccccctgggcctatactgaccctcatgctcgaaagcgtgaggacccaaccggattagatgccctgataggccatgccccacaccatgccatgtgttaggggcccatttccttagggaggcagctatggggaattttggacaatgtgggaaaccctgatccaacaatgccgcgtgtgtgaataaggccttcgggttgtaaagcacttttatccggatagattccttttgggctaaacctccgtaggggatgacggtaccggaagaataaccaccgggtaactacgtgccagcagccgcggtaatacgtagggtgcgagcgttaatcggaattactgggcgtaaagcgtgcgcaggcggtttgttaagacagatgtgaaatccccgggcttaacctgggaactgcatttgtgactggcaagctagagtatggcagacgggggtagaattccacgtgtagcagtgaaatgcgtagagatgtggaggaataccgatgggcgaagcagctcctggggcaatactgacgctcatgcacaagatcgtgcgaaacaaacaggataaaacccctgtattccacgcccaaaacgatgtccaccaagttgttggcgatcctttccttcgtatcgtagctacgcgggaatttgaccccctggggactaggccgcatataaaactcaagggaattccggggacccccagagctgtgtatgatgtgattattccgatgcgcggaaaaccttccttatctttgaatggcggtactcctgaaaattgcggagtgctcgaaaacaccgaacccgggtctttctgcgtgtcctccctcgtgtgggatatgctggatatcccgcagacgcatctttgacttagtgctcccaaaactgagagctgggaggactcgagaggggatccctgcctccccggcttgggtgctccccttatgggggaaacaggtacacggggggatcatcccataccta

SEQ ID NO: 12 - DNA, 1067 bp, Burkholderia
  strain A396
tctaaggagactgccggtgacaaaccggaggaaggtggggatgacgtcaagtcctcatggcccttatgggtagggcttcacacgtcatacaatggtcggaacagagggttgccaacccgcgagggggagctaatcccagaaaaccgatcgtagtccggattgcactctgcaactcgagtgcatgaagctggaatcgctagtaatcgcggatcagcatgccgcggtgaatacgttcccgggtcttgtacacaccgcccgtcacaccatgggagtgggttttaccagaagtggctagtctaaccgcaaggaggacggtcaccacggtaggattcatgactggggtgaagtcgtaacaaggtagccgtatcggaaggtgcggctggatcacctccttaaaccctttggcctaataaccccgggggaataagtaccgaaaaaaaaaaaaactggataacttccgtgccacaacccgcggaaaaatctagggggggggagcttaaatggaaatttacggggccgtaaagcgtgcgcaggcggtttgtaaacacagatgtgaaatccccgggcttaacctgggaactgcatttgtgactggcaagctagagtatggcacaggggggtagaattccacgtgtagcattgaatgcatagagatgagaggataccgatggagaagggcgcccccggggacaatatgacgcctatgccacaaagctgtggcacaataggttaaatacctgtgttgtccccgcctaaacagattacacttgttgtgggtattttctcataaaatactacacacgggagaatacactggggggcttcgtcaattatcacaacaatgattgcgggcacccacgggggtagatgggtaataaatcgacggcaactatctacttacttggatgatcgcacagattgggcgggagagaagagaacagcgtgtgtgtgctcctccgcgagtgataggtaatcggacaatactttgacaggacttaactgggtagcgggatcgagtggattcccgtcggatggcctccgcaggtacggcagctggggattacatc

SEQ ID NO: 13 - DNA, 1223 bp, Burkholderia
  strain A396
ttgcttacgacttcaccccagtcatgaatcctaccgtggtgaccgtcctccttgcggttagactagccacttctggtaaaacccactcccatggtgtgacgggcggtgtgtacaagacccgggaacgtattcaccgcggcatgctgatccgcgattactagcgattccagcttcatgcactcgagttgcagagtgcaatccggactacgatcggttttctgggattagctccccctcgcgggttggcaaccctctgttccgaccattgtatgacgtgtgaagccctacccataagggccatgaggacttgacgtcatccccaccttcctccggtttgtcaccggcagtctccttagagtgctcttgcgtagcaactaaggacaagggttgcgctcgttgcgggacttaacccaacatctcacgacacgagctgacgacagccatgcagcacctgtgtatcggttctctttcgagcactcccgaatctcttcaggattccgaccatgtcaagggtaggtaaggtttttcgcgttgcatcgaattaatccacatcatccaccgcttgtgcgggtccccgtcaattcctttgagttttaatcttgcgaccgtactccccaggcggtcaacttcacgcgttagctacgttactaaggaaatgaatccccaacaactagttgacatcgtttagggcgtggactaccagggtatctaatcctgtttgctccccacgctttcgtgcatgagcgtcagtattggcccagggggctgccttcgccatcggtattcctccacatctctacgcatttcactgctacacgtggaattctacccccctctgccatactctagcttgccagtcacaaatgcagttcccaggttaagcccggggatttcacatctgtcttaacaaaccgcctgcgcacgctttacgcccagtaattccgattaacgctcgcaccctacgtattaccgcggctgctggcacgtagttagccggtgcttattctgcggtaccgtcatcccccgggtatagcccaaaggattctttcgacaaagtgctttacacccgatgtctctcacacacgcgcatgctgatcaggtttccccatgtcaaagtccactgctgctcgtaggtctggacgggttcagttcaatgtgactgatcgtctttcgacaactactgaacgtccctgtagcttacccaccaactagctatagcatgc

SEQ ID NO: 14 - DNA, 1216 bp, Burkholderia
  strain A396
ccgagctgacgacagccatgcagcacctgtgtatcggttctctttcgagcactcccgaatctcttcaggattccgaccatgtcaagggtaggtaaggtttttcgcgttgcatcgaattaatccacatcatccaccgcttgtgcgggtccccgtcaattcctttgagttttaatcttgcgaccgtactccccaggcggtcaacttcacgcgttagctacgttactaaggaaatgaatccccaacaactagttgacatcgtttagggcgtggactaccagggtatctaatcctgtttgctccccacgctttcgtgcatgagcgtcagtattggcccagggggctgccttcgccatcggtattcctccacatctctacgcatttcactgctacacgtggaattctacccccctctgccatactctagcttgccagtcacaaatgcagttcccaggttaagcccggggatttcacatctgtcttaacaaaccgcctgcgcacgctttacgcccagtaattccgattaacgctcgcaccctacgtattaccgcggctgctggcacgtagttagccggtgcttattcttccggtaccgtcatccccccggggtattagcccaaaggatttctttccggacaaaagtgctttacaacccgaaggccttcttcacacacgcggcattgctggatcagggtttcccccattgtccaaaattccccactgctgcctcccgtaggagtctgggccgtgtctcagtcccagtgtggctgatcgtcctctcagaccagctactgatcgtcgccttggtaggcctttaccccaccaactagctaatcagccatcggccaaccctatagcgcgaggtccgaagatcccccgctttcatccgtagatcgtatgcggtattaatccggctttcgccgggctatcccccactacaggacatgttccgatgtattactcacccgttcgccactcgccccaggtgcaagcacccgtgctgccgttcgacttgcatgtgtagcatgcgcagcgtcatctactaaataaacaactctaagaatttttgcccgagggcctctaaacactcggggcgtcgagagagactacggatgaggagcatccctctgtctctaggtatgtgttgtcgcctctctcacagaggaggggacgcacgacggagccatcggggacgacaacatgtacgatatactatcta

SEQ ID NO: 15 - DNA, 1194 bp, Burkholderia
  strain A396
ttcttcggtaccgtcatccccccggggtattagcccaaaggatttctttccggacaaaagtgctttacaacccgaaggccttcttcacacacgcggcattgctggatcagggtttcccccattgtccaaaattccccactgctgcctcccgtaggagtctgggccgtgtctcagtcccagtgtggctgatcgtcctctcagaccagctactgatcgtcgccttggtaggcctttaccccaccaactagctaatcagccatcggccaaccctatagcgcgaggtccgaagatcccccgctttcatccgtagatcgtatgcggtattaatccggctttcgccgggctatcccccactacaggacatgttccgatgtattactcacccgttcgccactcgccaccaggtgcaagcacccgtgctgccgttcgacttgcatgtgtaaggcatgccgccagcgttcaatctgagccatgatcaaactctgagggggggggccttcaacggaacgactgggcaaaaagcgtgcccaggcgttttgttaagacagatgtgaaaccccggggcttaacctggaaactgcatttgtgactggaaagctagagtatggcagaggggggtagaattccacgtgtagcattgaaatgcgtagaaatggagaggaataccgatgggagagggcagcccccgtgggcaaatactggcgcttatgaacaaagttggggcgcgccgccgggatatgttcccctgggatatcccccccctaaactgcttacaaatattgtgtgggaaactttttctctaaaaaatagaacacaacgggagatatcacccccggggggccaccgccagattaaacccccaaaaagtatttggcgggcacccccccggggggtgagatggggtaaaataaatccgtgcgacgagcaaaccctccccacacctgggatggtcgcgaccacagatgagatgcgggcggagagaacgatacccaagcgtggttgtttgcctgcatcccctccgtcgggagtggatatagtagagtaattacggcacgactgcatttttttttcttcagtacaccttatcacactgttggatgcaccgcgagaaatccggaggtgtgagtactccccccctctcctcgggatgtgtcggcgctcccttctcccgttcaggggtgggtaagcaccgcg